(19) United States
(12) Patent Application Publication
(10) Pub. No.: US 2013/0210146 A1
(43) Pub. Date: Aug. 15, 2013

(54) Methods and Compositions for Generation of Induced Pluripotent Stem Cells By RNAA

(71) Applicant: Long-Cheng Li, San Francisco, CA (US)

(72) Inventor: Long-Cheng Li, San Francisco, CA (US)

(73) Assignee: THE REGENTS OF THE UNIVERSITY OF CALIFORNIA, Oakland, CA (US)

(21) Appl. No.: 13/817,091

(22) PCT Filed: Aug. 24, 2011
(86) PCT No.: PCT/US11/48985

§ 371 Date: Apr. 8, 2013

Related U.S. Application Data
(60) Provisional application No. 61/377,259, filed on Aug. 26, 2010.

(51) Int. Cl. C12N 5/071

(52) U.S. Cl. CPC C12N 5/0696
USPC 435/377; 536/24.1

(57) ABSTRACT

The present disclosure provides methods for inducing somatic cells to form induced pluripotent stem (iPS) cells. The method includes introducing a small activating RNA (saRNA) molecule into the somatic cell, where the saRNA molecule increases transcription of a transcription factor that induces the formation of induced pluripotent stem cells. The present disclosure also provides compositions and kits comprising a saRNA molecule that increases transcription of a transcription factor that induces the formation of induced pluripotent stem cells. The present disclosure provides iPS cells comprising at least one exogenous saRNA molecule, where the saRNA molecule increases transcription of a transcription factor that induces the formation of induced pluripotent stem cells.

### CROSS-REFERENCE TO EARLIER FILED APPLICATION

Pursuant to 35 U.S.C. §119(e), this application claims priority to U.S. Provisional Patent Application Ser. No. 61/377,259, filed Aug. 26, 2010, which application is incorporated herein by reference in its entirety.

### GOVERNMENT RIGHTS

This invention was made with government support under grant no. R01GM090293 awarded by National Institutes of Health. The United States Government has certain rights in this invention.

### INTRODUCTION

Embryonic stem (ES) cells, derived from the inner cell mass of blastocysts, are pluripotent and self-renewing cells, with the ability to give rise to all three germ layers-ectoderm, mesoderm, and endoderm. ES cells are thus valuable in regenerative medicine for the treatment of various types of diseases. However, therapeutic use of human ES cells is complicated by ethical controversy because early human embryos are destroyed in order to obtain the ES cells. There are also concerns of immune rejection associated with transplanting of ES cells into a subject.

Reprogramming of mouse and human somatic cells into pluripotent stem cells (referred to as induced pluripotent stem (iPS) cells) is of great interest, especially, to circumvent the need for ES cells to generate pluripotent stem cells. Viral transduction of transcription factors (transcription factor s) has been shown to result in the production of iPS cells from somatic cells (Takahashi K, Yamanaka S, Cell 2006; 126: 663-76; Wernig M, et al., Nature 2007; 448: 318-24; Maherali N, et al., Cell 2007; 1:70; Takahashi K, et al., Cell 2007; 131:861-72; Park I H, et al., Nature, 2008 Jan. 10, 451(7175):141-6.2007; Yu J et al., Human Somatic Cells, Science, 2007, Dec. 21; 318(5858):1917-20). These advances have firmly established proof of principle for generating pluripotent stem cells without destroying embryos, and hold great potential for application in regenerative medicine. Despite these advantages, the requirement of viral vectors in these methods prevents them from clinical use due to potential adverse effects associated with the use of viral vectors such as insertional mutagenesis.

There is a need for compounds that can reprogram somatic cells into pluripotent stem cells to form iPS cells. The present invention addresses these needs, as well as others.

### SUMMARY

The present disclosure provides methods for inducing somatic cells to form induced pluripotent stem cells by RNA activation (RNAa). The method includes introducing a small activating RNA (saRNA) molecule into the somatic cell, where the saRNA molecule increases transcription of a transcription factor that induces the formation of induced pluripotent stem cells. The present disclosure also provides compositions and kits comprising a saRNA molecule that increases transcription of a transcription factor that induces the formation of iPS cells. The present disclosure provides iPS cells comprising at least one exogenous saRNA molecule, where the saRNA molecule increases transcription of a transcription factor that induces the formation of induced pluripotent stem cells.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1, panels A-C illustrate that saRNAs induce potent and prolonged transcriptional gene activation.

FIG. 2 shows a schematic of the Oct4 promoter.

FIG. 3 illustrates that promoter-targeted saRNAs induce Oct4 mRNA expression in HeLa cells.

FIG. 4 depicts Oct4 activation by saRNAs in IMR-90 fibroblast cells.

FIG. 5, panel A depicts vector map of the lentiviral pGreenZeo-MYC reporter. FIG. 5, panel B illustrates results from flow cytometry analysis. FIG. 5, panel C depicts GFP expression in BJ fibroblast cells. FIG. 5, panel D depicts qRT-PCR data.

FIG. 6, panels A-D illustrate that saRNA restores the natural function of KLF4 as a transcription factor.

FIG. 7, panels A and B depict NANOG activation in NCCIT cells.

FIG. 8, panels A-C illustrate that saRNA increases iPS reprogramming efficiency.

FIG. 9, panel A depicts the location of Oct4 promoter-specific saRNA and the promoter sequence present between positions −628 to −598 relative to Oct4 transcription start site. FIG. 9, panels B-E illustrate the effect of dsOct4 molecules on Oct4 mRNA expression in human adipose-derived stem cells (hADSCs).

FIG. 10 shows a schematic of iPS cell induction protocol for hADSCs.

FIG. 11 illustrates formation of iPS cells using Oct4 promoter-specific saRNA.

FIG. 12 illustrates expression of Tra-1-60 in iPS cells obtained using Oct4 promoter-specific saRNA.

### DETAILED DESCRIPTION OF THE INVENTION

The present disclosure provides compositions, pharmaceutical preparations, and methods for producing a pluripotent stem cell from a somatic cell by contacting the somatic cells with a small activating RNA (saRNA) molecule comprising a ribonucleic strand that is complementary to a promoter region sequence of a gene encoding a transcription factor, which reprograms the somatic cell into a pluripotent stem cell. Also provided are kits for practicing the subject methods of the invention

Before the present invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a molecule” includes a plurality of such molecules and reference to “the molecule” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

As used herein the term “isolated” is meant to describe a compound of interest (e.g., either a polynucleotide or a polypeptide) that is in an environment different from that in which the compound naturally occurs.

“Purified” as used herein refers to a compound removed from an environment in which it was produced and is at least 60% free, preferably 75% free, and most preferably 90% free from other components with which it is naturally associated or with which it was otherwise associated with during production.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

Perfect complementarity or 100% complementarity refers to the situation in which each nucleotide unit of one polynucleotide strand can hydrogen bond with a nucleotide unit of a second polynucleotide strand, without a “mismatch”. Less than perfect complementarity refers to the situation in which not all nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to about 79%, about 80%, about 85%, about 90%, about 95%, or greater complementarity. Thus, for example, two polynucleotides of 29 nucleotide units each, wherein each comprises a di-dT at the 3′ terminus such that the duplex region spans 27 bases, and wherein 27 of the 27 bases of the duplex region on each strand are complementary, are substantially complementary. In determining complementarity, overhang regions are excluded.

The term “conjugate” refers to a polynucleotide that is covalently or non-covalently associated with a molecule or moiety that alters the physical properties of the polynucleotide, such as increasing stability and/or facilitate cellular uptake of a double stranded RNA, for example, but does not significantly affect the ability of the polynucleotide to base pair with a complementary polynucleotide. A “terminal conjugate” may have a molecule or moiety attached directly or indirectly through a linker to a 3′ and/or 5′ end of a polynucleotide or double stranded polynucleotide. An internal conjugate may have a molecule or moiety attached directly or indirectly through a linker to a base, to the 2′ position of the ribose, for example, or to other positions that do not interfere with Watson-Crick base pairing, for example, 5-aminoallyl uridine.

In a double stranded polynucleotide, one or both 5′ ends of the strands of polynucleotides comprising the double stranded polynucleotide can bear a conjugated molecule or moiety, and/or one or both 3′ ends of the strands of polynucleotides comprising the double stranded polynucleotide can bear a conjugated molecule or moiety.

Conjugates may contain, for example, amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates are steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-5-tritylthiol, thiocholesterol, acyl chains such as dodecandiol or undecyl groups, phospholipids such as di-hexadecyl-rac-glycerol, triethylammonium 1,2-di-O-hexadecyl-rac-glycer-o-3-H-phosphonate, polyamines, polyethylene glycol, adamantane acetic acid, palmityl moieties, octadecylamine moieties, hexylaminocarbonyl-oxyc-holesterol, farnesyl, geranyl and geranylgeranyl moieties.

Conjugates can also comprise a detectable label. For example, conjugates can be a polynucleotide covalently attached to a fluorophore. Conjugates may include fluorophores such as TAMRA, BODIPY, Cyanine derivatives such as Cy3 or Cy5, Dabsyl, or any other suitable fluorophore known in the art.

A conjugate molecule or moiety may be attached to any position on the terminal nucleotide that is convenient and that does not substantially interfere with the desired activity of the polynucleotide(s) that bear it, for example the 3′ or 5′ position of a ribosyl sugar. A conjugate molecule or moiety substantially interferes with the desired activity of a saRNA if it adversely affects its functionality such that the ability of the saRNA to mediate gene activation is reduced, for example, by greater than 80% in an in vitro assay employing cultured cells, where the functionality is measured at 24 hours post transfection.

The phrase “effective concentration” refers to a concentration of saRNA in a cell effective to cause an increase in transcription of a gene of interest in the cell. Of particular interest is an effective concentration that provides a greater than or equal to at least about 10% or more, 20% or more, 30% or more, 45% or more increase, including about 50% or more, about 60% or more, about 70% or more, about 75% or more, about 80% or more increase in target sequence activity relative to a basal expression level at 24, 48, 72, or 96 hours following administration. Target sequence activity may be measured by any method known in the art. For example, where the target sequence is a promoter, target sequence activity may be measured by level of transcription, level of the protein whose transcription is operably linked or operably associated with the promoter, or activity of the protein whose transcription is operably linked or operably associated with the promoter.

The term “polynucleotide” refers to polymers of nucleotides, and includes but is not limited to single stranded or double stranded molecule of DNA, RNA, or DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the substitution or attachment of various entities or moieties to the nucleotide units at any position, as well as naturally-occurring or non-naturally occurring backbones, are included.

The term “polyribonucleotide” refers to a polynucleotide comprising two or more modified or unmodified ribonucleotides and/or their analogs.

The phrases “operably associated” and “operably linked” refer to functionally related nucleic acid sequences. By way of example, a regulatory sequence is operably linked or operably associated with a protein encoding nucleic acid sequence if the regulatory sequence can exert an effect on the expression of the encoded protein. In another example, a promoter is operably linked or operably associated with a protein encoding nucleic acid sequence if the promoter controls the transcription of the encoded protein. While operably associated or operably linked nucleic acid sequences can be contiguous with the nucleic acid sequence that they control, the phrases “operably associated” and “operably linked” are not meant to be limited to those situations in which the regulatory sequences are contiguous with the nucleic acid sequences they control.

The term “gene” as used herein includes sequences of nucleic acids that when present in an appropriate host cell facilitates production of a gene product. “Genes” can include nucleic acid sequences that encode proteins, and sequences that do not encode proteins, and includes genes that are endogenous to a host cell or are completely or partially recombinant (e.g., due to introduction of an exogenous polynucleotide encoding a promoter and a coding sequence, or introduction of a heterologous promoter adjacent an endogenous coding sequence, into a host cell). For example, the term “gene” includes nucleic acid that can be composed of exons and introns. Sequences that code for proteins are, for example, sequences that are contained within exons in an open reading frame between a start codon and a stop codon. “Gene” as used herein refers to a nucleic acid that includes, for example, regulatory sequences such as promoters, enhancers and all other sequences known in the art that control the transcription, expression, or activity of a nucleic acid sequence operably linked or operably associated to the regulatory sequence, whether the nucleic acid sequence comprises coding sequences or non-coding sequences. In one context, for example, “gene” may be used to describe a nucleic acid comprising regulatory sequences such as promoter or enhancer and coding and non-coding sequences. The expression of a recombinant gene may be controlled by one or more heterologous regulatory sequences. “Heterologous” refers to two elements that are not normally associated in nature.

A “target gene” is a nucleic acid containing a sequence, such as, for example, a promoter or enhancer, against which a saRNA can be directed for the purpose of affecting activation of expression. Either or both “gene” and “target gene” may be nucleic acid sequences naturally occurring in an organism, transgenes, viral or bacterial sequences, chromosomal or extrachromosomal, and/or transiently or chronically transfected or incorporated into the cell and/or its chromatin. A “target gene” can, upon saRNA-mediated activation, repress the activity of another “gene” such as a gene coding for a protein (as measured by transcription, translation, expression, or presence or activity of the gene's protein product). In another example, a “target gene” can comprise an enhancer, and saRNA mediated activation of the enhancer may increase the functionality of an operably linked or operably associated promoter, and thus increase the activity of another “gene” such as a gene coding for a protein that is operably linked to the increased promoter and/or enhancer.

“Regulatory elements” are nucleic acid sequences that regulate, induce, repress, or otherwise mediate the transcription, translation of a protein or RNA coded by a nucleic acid sequence with which they are operably linked or operably associated. Typically, a regulatory element or sequence such as, for example, an enhancer or repressor sequence, is operatively linked or operatively associated with a protein or RNA coding nucleic acid sequence if the regulatory element or regulatory sequence mediates the level of transcription, translation or expression of the protein coding nucleic acid sequence in response to the presence or absence of one or more regulatory factors that control transcription, translation and/or expression. Regulatory factors include, for example, transcription factors. Regulatory sequences may be found in introns.

Regulatory sequences or elements include, for example, “TATAA” boxes, “CAAT” boxes, differentiation-specific elements, cAMP binding protein response elements, sterol regulatory elements, serum response elements, glucocorticoid response elements, transcription factor binding elements such as, for example, SPI binding elements, and the like. A “CAAT” box is typically located upstream (in the 5′ direction) from the start codon of a eukaryotic nucleic acid sequence encoding a protein or RNA. Examples of other regulatory sequences include splicing signals, polyadenylation signals, termination signals, and the like. Further examples of nucleic acid sequences that comprise regulatory sequences include the long terminal repeats of the Rous sarcoma virus and other retroviruses. An example of a regulatory sequence that controls tissue-specific transcription is the interferon-epsilon regulatory sequence that preferentially induces production of the operably linked sequence encoding the protein in placental, tracheal, and uterine tissues, as opposed to lung, brain, liver, kidney, spleen, thymus, prostate, testis, ovary, small intestine, and pancreatic tissues. Numerous regulatory sequences are known in the art, and the foregoing is merely illustrative of a few.

The term “enhancer” and phrase “enhancer sequence” refer to a variety of regulatory sequence that can increase the efficiency of transcription, without regard to the orientation of the enhancer sequence or its distance or position in space from the promoter, transcription start site, or first codon of the nucleic acid sequence encoding a protein with which the enhancer is operably linked or associated.

The term “promoter” refers to a nucleic acid sequence that does not code for a protein, and that is operably linked or operably associated to a protein coding or RNA coding nucleic acid sequence such that the transcription of the operably linked or operably associated protein coding or RNA coding nucleic acid sequence is controlled by the promoter. Typically, eukaryotic promoters comprise between 100 and 5,000 base pairs, although this length range is not meant to be limiting with respect to the term “promoter” as used herein. Although typically found 5′ to the protein coding nucleic acid sequence to which they are operably linked or operably associated, promoters can be found in intron sequences as well. The term “promoter” is meant to include regulatory sequences operably linked or operably associated with the same protein or RNA encoding sequence that is operably linked or operably associated with the promoter. Promoters can comprise many elements, including regulatory elements. The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent.

The phrase “non-coding target sequence” or “non-coding nucleic acid sequence” refers to a regulatory nucleic acid sequence of interest that is not contained within an exon of a gene. Examples of “non-coding target sequence” or “non-coding nucleic acid sequence” include promoter regions, enhancer regions, and the like.

“Nucleotide analogs” include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

“Modified bases” refer to nucleotide bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

The phrase “nucleotide unit” refers to a single nucleotide residue and is comprised of a modified or unmodified nitrogenous base, a modified or unmodified sugar, and a modified or unmodified moiety that allows for linking of two nucleotides together or a nucleotide to a conjugate that precludes further linkage. The single nucleotide residue may be in a polynucleotide. Thus, a polynucleotide having 27 bases has 27 nucleotide units.

The phrase “nuclear uptake enhancing modification” refers to a modification of a naturally occurring or non-naturally occurring polynucleotide that provides for enhanced nuclear uptake of the modified polynucleotide. An example of a “nuclear uptake enhancing modification” is a stabilizing modification, such as a modified internucleotide linkage, that confers sufficient stability on a molecule, such as a nucleic acid, to render it sufficiently resistant to degradation (e.g., by nucleases) such that the associated nucleic acid can accumulate in the nucleus of a cell when exogenously introduced into the cell. In this example, entry into the cell's nucleus is facilitated by the ability of the modified nucleic acid to resist nucleases sufficiently well such that an effective concentration of the nucleic acid can be achieved inside the nucleus. An effective concentration is a concentration that results in a detectable change in the transcription or activity of a gene or target sequence as the result of the accumulation of nucleic acid within the nucleus.

The phrases “orthoester protected” and “orthoester modified” refer to modification of a sugar moiety within a nucleotide unit with an orthoester. Preferably, the sugar moiety is a ribosyl moiety. In general, orthoesters have the structure RC(OR′)₃ wherein each R′ can be the same or different, R can be an H, and wherein the underscored C is the central carbon of the orthoester. The orthoesters of the present invention are comprised of orthoesters wherein a carbon of a sugar moiety in a nucleotide unit is bonded to an oxygen, which is in turn bonded to the central carbon of the orthoester. To the central carbon of the orthoester is, in turn, bonded two oxygens, such that in total three oxygens bond to the central carbon of the orthoester. These two oxygens bonded to the central carbon (neither of which is bonded to the carbon of the sugar moiety) in turn, bond to carbon atoms that comprise two moieties that can be the same or different. For example, one of the oxygens can be bound to an ethyl moiety, and the other to an isopropyl moiety. In one example, R can be an H, one R′ can be a ribosyl moiety, and the other two R′ moieties can be 2-ethyl-hydroxyl moieties. Orthoesters can be placed at any position on the sugar moiety, such as, for example, on the 2′, 3′ and/or 5′ positions. Exemplary orthoesters, and methods of making orthoester protected polynucleotides, are described in U.S. Pat. Nos. 5,889,136 and 6,008,400, each herein incorporated by reference in its entirety.

The term “stabilized” refers to the ability of a saRNA to resist degradation while maintaining functionality and can be measured in terms of its half-life in the presence of, for example, biological materials such as serum. The half-life of a saRNA, for example, in serum refers to the time taken for the 50% of saRNA to be degraded.

The phrase “duplex region” refers to the region in two complementary or substantially complementary polynucleotides that form base pairs with one another, either by Watson-Crick base pairing or any other manner that allows for a duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” consists of 19 base pairs. The remaining base pairs may, for example, exist as 5′ and 3′ overhangs. Further, within the duplex region, 100% complementarity is not required; substantial complementarity is allowable within a duplex region. Substantial complementarity generally refers to about at least 79%, about 80%, about 85%, about 85%, about 90%, about 95% or greater complementarity. For example, a mismatch in a duplex region consisting of 19 base pairs (i.e., 18 base pairs and one mismatch) results in about 94.7% complementarity, rendering the duplex region substantially complementary. In another example, three mismatches in a duplex region consisting of 19 base pairs (i.e., 16 base pairs and three mismatches) results in about 84.2% complementarity, rendering the duplex region substantially complementary, and so on.

The term “overhang” refers to a terminal (5′ or 3′) non-base pairing nucleotide resulting from one strand extending beyond the other strand within a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

As used herein, the terms “gene activating”, “activating a gene”, or “gene activation” are interchangeable and refer to an increase in transcription, translation or expression or activity of a nucleic acid, as measured by transcription level, e.g., mRNA level; enzymatic activity; methylation state; chromatin state or configuration; translational level; or other measure of its activity or state in a cell or biological system. Such activities or states can be assayed directly or indirectly. Furthermore, “gene activating”, “activating a gene”, or “gene activation” refer to the increase of activity associated with a nucleic acid sequence, such as its ability to function as a regulatory sequence, its ability to be transcribed and result in expression of a protein, regardless of the mechanism whereby such activation occurs.

The phrase “pharmaceutically acceptable carrier” refers to compositions that facilitate the introduction of dsRNA into a cell and includes but is not limited to solvents or dispersants, coatings, anti-infective agents, isotonic agents, agents that mediate absorption time or release of the inventive polynucleotides and double stranded polynucleotides. Examples of “pharmaceutically acceptable carriers” include liposomes that can be neutral or cationic, can also comprise molecules such as chloroquine and 1,2-dioleoyl-sn-glycero-3-phosphatidyle-thanolamine, which can help destabilize endosomes and thereby aid in delivery of liposome contents into a cell, including a cell's nucleus. Examples of other pharmaceutically acceptable carriers include poly-L-lysine, polyalkylcyanoacrylate nanoparticles, polyethyleneimines, and any suitable PAMAM dendrimers (polyamidoamine) known in the art with various cores such as, for example, ethylenediamine cores, and various surface functional groups such as, for example, cationic and anionic functional groups, amines, ethanolamines, aminodecyl.

The term “saRNA” refers to a small activating RNA molecule that includes at least a first ribonucleic acid strand comprising a ribonucleic acid sequence at least 80% complementary to a promoter region sequence of a target gene. saRNA are may be double stranded RNA duplex. The saRNA can also be provided as single stranded molecule that forms a double-stranded region with a hairpin loop, wherein a first sequence forming the double stranded region is a ribonucleotide sequence complementary to a promoter region sequence of a target gene, and a second sequence forming the double strand comprises a ribonucleotide sequence complementary to the first sequence and forming a duplex region with the first region. saRNA are generally about up to 30 nucleotides long, such as, 15 nucleotides-30 nucleotides long, for example, 17 nucleotides-27 nucleotides long, or 19 nucleotides-25 nucleotides long, or 19 nucleotides-21 nucleotides long, e.g., 15 nucleotides, 19 nucleotides, 21 nucleotides, 25 nucleotides, 27 nucleotides, or 30 nucleotides.

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, fetal, post-natal, juvenile or adult tissue.

The term “progenitor cell”, as used herein, refers to an undifferentiated cell derived from a stem cell, and is not itself a stem cell. Some progenitor cells can produce progeny that are capable of differentiating into more than one cell type.

The term “induced pluripotent stem cell” (or “iPS cell”), as used herein, refers to a stem cell induced from a somatic cell (e.g., a differentiated somatic cell) or a multi-potent cell (e.g., placenta-derived mesenchymal stem cells, adipose-derived stem cells (ADSCs), hematopoietic cell), and that has a higher potency than the somatic cell or multi-potent cell. iPS cells are capable of self-renewal and differentiation into mature cells. iPS may also be capable of differentiation into progenitor cells that can produce progeny that are capable of differentiating into more than one cell type.

### DETAILED DESCRIPTION

The present disclosure provides methods for inducing somatic cells to form induced pluripotent stem cells. The method includes introducing a small activating RNA (saRNA) molecule into the somatic cell, where the saRNA molecule increases transcription of a transcription factor that induces the formation of induced pluripotent stem cells. The present disclosure also provides compositions and kits comprising a saRNA molecule that increases transcription of a transcription factor that induces the formation of induced pluripotent stem cells.

### Methods

As noted above a method for inducing a somatic cell to form an induced pluripotent stem (iPS) cell is provided. The method includes introducing into the somatic cell a saRNA molecule, wherein the saRNA molecule comprises a first ribonucleic acid strand comprising a ribonucleic acid sequence at least 80% complementary to a promoter region sequence of a transcription factor gene, wherein the transcription factor gene encodes a transcription factor that induces the somatic cell to form a pluripotent stem cell, wherein the introducing is sufficient to activate the transcription of the transcription factor gene; and culturing the somatic cell to produce an induced pluripotent stem cell.

In certain embodiments, the ribonucleic acid sequence at least 80% complementary to a promoter region sequence of a transcription factor gene may be at least 85% complementary, or at least 90% complementary, or at least 95% complementary, or at least 99% complementary, or 100% complementary to the promoter region sequence.

In certain embodiments, the saRNA molecule comprising a first ribonucleic acid strand comprising a ribonucleic acid sequence at least 80% complementary to a promoter region sequence of a transcription factor gene may include a 3′ terminal region of at least one nucleotide non-complementary to the promoter region sequence. In certain embodiments, the saRNA molecule may comprise a first ribonucleic acid strand comprising: a 5′ region of complementarity to a promoter region sequence of a transcription factor gene, wherein the transcription factor gene encodes a transcription factor that induces the somatic cell to form a pluripotent stem cell and a 3′ terminal region of at least one nucleotide non-complementary to the promoter region sequence.

The transcription factor gene that encodes a transcription factor that induces the somatic cell to form a pluripotent stem cell may be any transcription factor that can induce a somatic cell to form a pluripotent stem cell. Examples of such transcription factors include, an Oct family gene, a Sox family gene, a Myc family gene, a Klf family gene, a Nanog family gene, a Lin28 family gene or a NR5A (nuclear receptor subfamily 5, group A) family gene. Examples of transcription factor genes from the Oct family include Oct4, Oct1A, Oct6, and the like. Examples of transcription factor genes from the Sox family include Sox1, Sox-2, Sox3, Sox7, Sox15, Sox17 and Sox18. Examples of the transcription factor genes from the Klf (Kruppel like factor-4) family gene include Klf1, Klf2, Klf4, Klf5 and the like. Examples of transcription factor genes from the Myc family gene include c-Myc, N-Myc, L-Myc and the like. Lin28 family genes include, for example, Lin28 and Lin28b. An example of transcription factor genes from the NR5A family is NR5A2.

The saRNA molecule is sufficient to activate transcription of the transcription factor gene and causes an increase in the expression of the transcription factor gene compared to a control, e.g., in the absence of the saRNA molecule or in the presence of an unrelated saRNA molecule. In representative embodiments, an increase in transcription factor gene expression results in a detectable transcription factor gene product while a control shows no detectable transcription factor gene product. In representative embodiments, an increase in transcription factor gene expression results in at least about a 2-fold increase or more in transcription associated with transcription factor gene sequence, as compared to a control. In some embodiments, the increase in transcription factor gene expression results in at least about a 2.5-fold increase or more, at least about a 3-fold increase or more, at least about a 3.5-fold increase or more, at least about a 4-fold increase or more, at least about a 4.5-fold increase or more, at least about a 5-fold increase or more, at least about a 5.5-fold increase or more, at least about a 6-fold increase or more, at least about a 6.5-fold increase or more, at least about a 7-fold increase or more, at least about a 7.5-fold increase or more at least about a 8-fold increase or more, and up to about 10-fold increase or more, including about 15-fold increase or more, about 20-fold increase or more, such as 25-fold increase or more in transcription associated with transcription factor gene sequence, as compared to a control. An increase in transcription associated with transcription factor gene sequence can be measured by any of a variety of methods well known in the art, for example, measuring transcription factor mRNA level, transcription factor c-DNA level, transcription factor protein level, transcription factor activity, and the like.

The somatic cell used to generate iPS cells may be a mammalian somatic cell. Examples of suitable mammalian somatic cells include, but are not limited to: fibroblasts (e.g., skin fibroblasts, dermal fibroblasts), bone marrow-derived mononuclear cells, muscle cells, peripheral blood mononuclear cells, macrophages, hepatocytes, keratinocytes, oral keratinocytes, hair follicle cells, dermal cells, epithelial cells, gastric epithelial cells, lung epithelial cells, synovial cells, kidney cells, skin epithelial cells, pancreatic beta cells, neuronal cell, retinal cell, glial cell, and osteoblasts, for example.

Mammalian somatic cells used to generate iPS cells can originate from a variety of types of tissue including but not limited to: bone marrow, skin (e.g., dermis, epidermis), muscle (e.g., skeletal muscle, cardiac muscle, smooth muscle), adipose tissue, peripheral blood, central nervous system tissue (e.g., brain, spinal cord). The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, greater than about 45 years old, greater than about 55 years old, greater than about 65 years old, or greater than about 80 years old.

The somatic cell may be obtained from mammals such as cattle, horse, goats, pigs, sheep, canines, rodents such as hamsters, mice, and rats, and primates such as, for example, gorillas, chimpanzees, and humans. Transgenic mammals may also be used, e.g. mammals that have a chimeric gene sequence.

The methods described herein may also be used convert a multipotent cell into an iPS cell. Examples of multipotent cells that can be induced to form iPS cells include placenta-derived mesenchymal stem cells, adipose-derived stem cells (ADSCs), and the like.

The introducing of the saRNA molecule into a cell may be carried out by a number of ways. For example, the polynucleotides may be passively delivered to cells. Passive uptake of modified polynucleotides can be modulated, for example, by the presence of a conjugate such as a polyethylene glycol moiety or a cholesterol moiety at the 5′ terminal of the sense strand.

The saRNA may be delivered to a cell by any method that is now known or that comes to be known and that from reading this disclosure, persons skilled in the art would determine would be useful in connection with the present invention in enabling saRNA to cross the cellular membrane and/or the nuclear membrane. These methods include, but are not limited to, any manner of transfection, such as for example transfection employing DEAE-Dextran, calcium phosphate, cationic lipids/liposomes, micelles, manipulation of pressure, microinjection, electroporation, immunoporation, use of vectors such as viruses (e.g., RNA virus), plasmids, cell fusions, and coupling of the polynucleotides to specific conjugates or ligands such as antibodies, antigens, or receptors, passive introduction (e.g., by the presence of a conjugate such as a polyethylene glycol moiety or a cholesterol moiety at the 5′ terminal of the sense strand), adding moieties to the saRNA that facilitate its uptake, and the like. One strategy for delivery of saRNA to a cell may be to use natural or synthetic lipophilic or polymeric carrier molecules. Certain natural and synthetic lipophilic molecules may also be organized into liposomes or particles as carriers for saRNA. saRNA may be incorporated into liposomes for delivery by a number of methods such as those described in U.S. Application publication 20100112042, U.S. Application Publication No. 20050175682, U.S. Application Publication No. 20060051405, for example. Liposomes may be synthesized by numerous methods such as those described in U.S. Application Publication No. 20080317839, U.S. Application Publication No. 20100130382, U.S. Application Publication No. 20050175682, U.S. Application Publication No. 20060051405, for example. All of the references cited herein are hereby incorporated by reference.

iPS cells produced by the methods disclosed herein may be detected based on the presence of one or more properties including but not limited to expression of particular proteins, an ES cell like morphology, pluripotency, growth properties, epigenetic reprogramming. These properties are described below. In certain embodiments, an iPS cell may possess two or more, or three or more, or four or more, or five or more, or six or more, or more, for example, seven of the following properties. iPS cells may produce and express on their cell surface one or more of the following cell surface antigens: Stage-Specific Embryonic Antigens 3 (SSEA-3) and 4 (SSEA-4), Tumor Rejection Antigens (TRA): TRA-1-60, TRA-1-81, TRA-2-49/6E (alkaline phophatase), and Nanog. iPS cells may express one or more of the following genes: Oct-3/4, Sox2, Nanog, GDF3, REX1, FGF4, ESG1, DPPA2, DPPA4, and hTERT. One or more of the foregoing cell surface markers or other genes expressed by iPS cells may be used to detect iPS cells. In certain embodiments, an iPS cell may be a cell that expresses at least two of the above-mentioned cell surface markers. In certain embodiments, an iPS cell may be a cell that expresses at least three of the above-mentioned cell surface markers. In certain embodiments, an iPS cell may be a cell that expresses at least three of the above-mentioned cell surface markers.

Detection of markers may be achieved through any means known in the art, for example immunologically. Histochemical staining, flow cytometry (FACS), Western Blot, enzyme-linked immunoassay (ELISA), etc. may be used. Flow immunocytochemistry may be used to detect cell-surface markers, immunohistochemistry (for example, of fixed cells or tissue sections) may be used for intracellular or cell-surface markers. Western blot analysis may be conducted on cellular extracts. Enzyme-linked immunoassay may be used for cellular extracts or products secreted into the medium. Antibodies for the identification of stem cell markers may be obtained from commercial sources, for example from Chemicon International, (Temecula, Calif., USA). The immunological detection of these antigens using monoclonal antibodies has been widely used to characterize pluripotent stem cells (Shamblott M J. et. al. (1998) PNAS 95: 13726-13731; Schuldiner M. et. al. (2000). PNAS 97: 11307-11312; Thomson J. A. et. al. (1998). Science 282: 1145-1147; Reubinoff B. E. et. al. (2000). Nature Biotechnology 18: 399-404; Henderson J. K. et. al. (2002). Stem Cells 20: 329-337; Pera M. et. al. (2000). J. Cell Science 113: 5-10.).

Other than gene expression, iPS cells may be detected by assessing cell morphology, pluripotency or multi-lineage differentiation potential or any characteristics known in the art, or a combination thereof.

The successfully generated iPS cells from previous studies were remarkably similar to naturally-isolated pluripotent stem cells (such as mouse and human embryonic stem cells, mESCs and hESCs, respectively), thus confirming the identity, authenticity, and pluripotency of iPS cells to naturally-isolated pluripotent stem cells. Thus, induced pluripotent stem cells generated from the methods disclosed herein could be selected based on one or more of following embryonic stem cell characteristics, as outlined below:

A. Cellular Biological Properties

Morphology: iPS cells are morphologically similar to ESCs. Each cell may have round shape, large nucleoli and scant cytoplasm. Colonies of iPS cells maybe also similar to that of ESCs. Human iPS cells form sharp-edged, flat, tightly-packed colonies similar to hESCs and mouse iPS cells form the colonies similar to mESCs, less flatter and more aggregated colonies than that of hESCs.

Growth properties: Doubling time and mitotic activity are cornerstones of ESCs, as stem cells must self-renew as part of their definition. iPS cells may be mitotically active, actively self-renewing, proliferating, and dividing at a rate equal to ESCs.

Stem Cell Markers: iPS cells may express cell surface antigenic markers expressed on ESCs. Human iPS cells expressed the markers specific to hESC, including, but not limited to, SSEA-3, SSEA-4, TRA-1-60, TRA-1-81, TRA-2-49/6E, and Nanog. Mouse iPS cells expressed SSEA-1 but not SSEA-3 nor SSEA-4, similarly to mESCs.

Stem Cell Genes: iPS cells may express genes expressed in undifferentiated ESCs, including Oct-3/4, Sox2, Nanog, GDF3, REX1, FGF4, ESG1, DPPA2, DPPA4, and hTERT.

Telomerase Activity: Telomerases are necessary to sustain cell division unrestricted by the Hayflick limit of 50 cell divisions. hESCs express high telomerase activity to sustain self-renewal and proliferation, and iPS cells also demonstrate high telomerase activity and express hTERT (human telomerase reverse transcriptase), a necessary component in the telomerase protein complex.

Pluripotency: iPS cells will be capable of differentiation in a fashion similar to ESCs into fully differentiated tissues.

Neural Differentiation: iPS cells could be differentiated into neurons, expressing P3 μ tubulin, tyrosine hydroxylase, AADC, DAT, ChAT, LMX1B, and MAP2. The presence of catecholamine-associated enzymes may indicate that iPS cells, like hESCs, may be differentiable into dopaminergic neurons. Stem cell-associated genes will be downregulated after differentiation.

Cardiac Differentiation: iPS cells could be differentiated into cardiomyocytes that spontaneously began beating. Cardiomyocytes expressed TnTc, MEF2C, MYL2A, MYHC β, and NKX2.5. Stem cell-associated genes will be downregulated after differentiation.

Teratoma Formation: iPS cells injected into immunodeficient mice may spontaneously formed teratomas after certain time, such as nine weeks. Teratomas are tumors of multiple lineages containing tissue derived from the three germ layers endoderm, mesoderm and ectoderm; this is unlike other tumors, which typically are of only one cell type. Teratoma formation is a landmark test for pluripotency.

Embryoid Body: hESCs in culture spontaneously form ball-like embryo-like structures termed “embryoid bodies,” which consist of a core of mitotically active and differentiating hESCs and a periphery of fully differentiated cells from all three germ layers. iPS cells may also form embryoid bodies and have peripheral differentiated cells.

Blastocyst Injection: hESCs naturally reside within the inner cell mass (embryoblast) of blastocysts, and in the embryoblast, differentiate into the embryo while the blastocyst's shell (trophoblast) differentiates into extraembryonic tissues. The hollow trophoblast is unable to form a living embryo, and thus it is necessary for the embryonic stem cells within the embryoblast to differentiate and form the embryo. iPS cells injected by micropipette into a trophoblast to generate a blastocyst transferred to recipient females, may result in chimeric living mouse pups: mice with iPS cell derivatives incorporated all across their bodies with 10%-90 and chimerism.

In certain embodiments, an iPS cell may be a cell that exhibits at least two of the above-mentioned cellular biological properties, for example, pluripotency and growth properties. In certain embodiments, an iPS cell may be a cell that exhibits at least three of the above-mentioned cellular biological properties, for example, pluripotency, growth properties, and embryoid body formation.

In certain embodiments, an iPS cell may be a cell that expresses at least one of the above mentioned cell surface markers and at least one of the above-mentioned cellular biological properties. For example, an iPS cell may be a cell that expresses SSEA-3, SSEA-4 and is pluripotent.

B. Epigenetic Reprogramming

Promoter Demethylation: Methylation is the transfer of a methyl group to a DNA base, typically the transfer of a methyl group to a cytosine molecule in a CpG site (adjacent cytosine/guanine sequence). Widespread methylation of a gene interferes with expression by preventing the activity of expression proteins or recruiting enzymes that interfere with expression. Thus, methylation of a gene effectively silences it by preventing transcription. Promoters of endogenous pluripotency-associated genes, including Oct-3/4, Rexl, and Nanog, may be demethylated in iPS cells, showing their promoter activity and the active promotion and expression of pluripotency-associated genes in iPS cells.

Histone Demethylation: Histones are compacting proteins that are structurally localized to DNA sequences that can affect their activity through various chromatin-related modifications. H3 histones associated with Oct-3/4, Sox2, and Nanog may be demethylated to activate the expression of Oct-3/4, Sox2, and Nanog.

In certain embodiments, an iPS cell may be a cell that expresses at least one of the above mentioned cell surface markers, at least one of the above mentioned cellular biological properties, and demethylation of promoter regions of endogenous pluripotency-associated genes or of histones associated with endogenous pluripotency-associated genes.

Culturing of iPS Cells. After somatic cells are introduced with saRNA molecules using the disclosed method, these cells may be cultured in a medium sufficient to maintain the pluripotency. Culturing of iPS cells can use various medium and techniques developed to culture pluripotent stem cells, more specially, embryonic stem cells, as described in U.S. Pat. App. 20070238170 and U.S. Pat. App. 20030211603.

For example, like human embryonic stem (hES) cells, iPS cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM .beta.-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM .beta.-mercaptoethanol. Just before use, human bFGF is added to a final concentration of .about 4 ng/mL (WO 99/20741).

iPS cells, like ES cells, have characteristic antigens that can be identified by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10 10 6 cells into the rear leg muscles of 8-12 week old male SCID mice. Teratomas develop that demonstrate at least one cell type of each of the three germ layers.

After the introduction of the saRNA molecule, the somatic cell may be cultured for about 1 hour, or about 3 hours, or about 10 hours, or about, 18 hours, or about 24 hours, or about 48 hours, or more to produce iPS cells. The somatic cell culture may be monitored at certain time points to detect the presence of iPS cells.

The iPS cells produced using the methods presented herein may be used for treatment of a subject in need of a stem cell therapy. In certain cases, the somatic cell may be isolated from the subject in need of therapy. In other cases, the somatic cell may be present in the subject and the saRNA may be introduced into the somatic cell by administering the saRNA to the subject. When administered to the subject, the saRNA may be present in a composition that targets the saRNA to a type of somatic cell.

Subjects receiving iPS cells or saRNA disclosed herein that result in the formation of iPS cells may be tested in order to assay the activity and efficacy of the subject saRNAs. Significant improvements in one or more of parameters are indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the compound administered, and the like).

As noted above, the saRNA molecule comprises a first ribonucleic acid strand comprising a ribonucleic acid sequence at least 80% complementary to a promoter region sequence of a transcription factor gene, wherein the transcription factor gene encodes a transcription factor that induces the somatic cell to form a pluripotent stem cell, wherein the introducing is sufficient to activate the transcription of the transcription factor gene. The saRNA molecule can be provided as a single stranded molecule. The saRNA molecule can also be provided as a double-stranded molecule, with a second strand complementary to the first strand and forming a duplex region with the first strand. In certain embodiments, the first and second strands may not be perfectly complementary in the region that forms the duplex. For example, the first and second strands may be 80%, 85%, 90%, 95%, or 99% complementary.

In certain embodiments, a one to two residue overhang at the 3′ ends of the first and/or the second strands may be present. The residue overhang at the 3′ ends of the first and/or the second strands may comprise any residue selected from A, G, C, U, or T. In certain cases, the residue overhang at the 3′ ends of the first and/or the second strands may comprise a nucleotide sequence: Xn, where n=0, 1, or 2, and X is A, G, C, U, or T.

The saRNA can also be provided as single stranded molecule that forms a double-stranded region with a hairpin loop, wherein a first sequence forming the double stranded region is a ribonucleotide sequence complementary to a promoter region sequence of a transcription factor gene, and a second sequence forming the double strand comprises a ribonucleotide sequence complementary to the first sequence and forming a duplex region with the first region. In certain embodiments, the first and second strands may not be perfectly complementary in the region that forms the duplex. For example, the first and second strands may be 80%, 85%, 90%, 95%, or 99% complementary. In certain embodiments, a two residue overhang at the 3′ ends of the strand may be present. For example, a single stranded saRNA may be made by joining the 3′ end of the sequence of SEQ ID NO: 1 to the 5′ end of the sequence of SEQ ID NO: 2 separated by a hairpin loop sequence, such as, AAU, AUAU, etc.

In certain embodiments, saRNA molecule may be up to 30 nucleotides long and may include a first ribonucleic acid strand, where the first ribonucleic acid strand is about 15 nucleotides-25 nucleotides, for example, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides long. In certain aspects, the saRNA molecule may be up to 30 nucleotides long and may include a first ribonucleic acid strand and a second ribonucleic acid strand that is complementary to the first ribonucleic acid strand.

In certain embodiments, the saRNA molecule may be up to 30 nucleotides long and may include a first ribonucleic acid strand comprising a sequence selected from the group consisting of SEQ ID NO: 1-136. In certain embodiments, the saRNA molecule may further comprise a second ribonucleic acid strand complementary (80%, 85%, 90%, 95%, 99%, or 100% complementary) to the first ribonucleic acid strand. In certain embodiments, the first and/or second ribonucleic acid strand may further comprise a one to five residue overhang at the 3′ end. In certain embodiments, saRNA molecule may comprise a thio modified internucleotide linkage.

Sequences of exemplary ribonucleic acid strands that may be included in the saRNA molecules disclosed herein are provided in Table 1 below.

TABLE 1
Sequences for saRNAs

| Name | Sequence (5′-3′) |
|---|---|
| dsOct4-141 | Sense: GCCACCACCAUUAGGCAAA |
| | (SEQ ID NO: 1) |
| | Antisense: UUUGCCUAAUGGUGGUGGC |
| | (SEQ ID NO: 2) |
| dsOct4-162 | Sense: UCCCCUUCCACAGACACCA |
| | (SEQ ID NO: 3) |
| | Antisense: UGGUGUCUGUGGAAGGGGA |
| | (SEQ ID NO: 4) |
| dsOct4-257 | Sense: AGU GAG ACC CUG UCU UAA A |
| | (SEQ ID NO: 5) |
| | Antisense: UUU AAG ACA GGG UCU CAC U |
| | (SEQ ID NO: 6) |
| dsOct4-275 | Sense: CUCCAGUCUGGGCAACAAA |
| | (SEQ ID NO: 7) |
| | Antisense: UUUGUUGCCCAGACUGGAG |
| | (SEQ ID NO: 8) |
| dsOct4-420 | Sense: GGCCCCAUCUCUACUAAAA |
| | (SEQ ID NO: 9) |
| | Antisense: UUUUAGUAGAGAUGGGGCC |
| | (SEQ ID NO: 10) |
| dsOct4-422 | Sense: AAG GCC CCA UCU CUA CUA A |
| | (SEQ ID NO: 11) |
| | Antisense: UUA GUA GAG AUG GGG CCU U |
| | (SEQ ID NO: 12) |
| dsOct4-573 | Sense: UGGGAUGUGCAGAGCCUGA |
| | (SEQ ID NO: 13) |
| | Antisense: UCAGGCUCUGCACAUCCCA |
| | (SEQ ID NO: 14) |
| dsOct4-581 | Sense: CAGACAGCUGGGAUGUGCA |
| | (SEQ ID NO: 15) |
| | Antisense: UGCACAUCCCAGCUGUCUG |
| | (SEQ ID NO: 16) |
| dsOct4-586 | Sense: GAUUCCAGACAGCUGGGAU |
| | (SEQ ID NO: 17) |
| | Antisense: AUCCCAGCUGUCUGGAAUC |
| | (SEQ ID NO: 18) |
| dsOct4-594 | Sense: GUGGGAGUGAUUCCAGACA |
| | (SEQ ID NO: 19) |
| | Antisense: UGUCUGGAAUCACUCCCAC |
| | (SEQ ID NO: 20) |
| dsOct4-598 | Sense: AGGUGUGGGAGUGAUUCCA |
| | (SEQ ID NO: 21) |
| | Antisense: UGGAAUCACUCCCACACCU |
| | (SEQ ID NO: 22) |
| dsOct4-616 | Sense: GGUUCCUGAAGAACAUGGA |
| | (SEQ ID NO: 23) |
| | Antisense: UCCAUGUUCUUCAGGAACC |
| | (SEQ ID NO: 24) |
| dsOct4-620 | Sense: CCUGGGUUCCUGAAGAACA |
| | (SEQ ID NO: 25) |
| | Antisense: UGUUCUUCAGGAACCCAGG |
| | (SEQ ID NO: 26) |
| dsOct4-622 | Sense: CACCUGGGUUCCUGAAGAA |
| | (SEQ ID NO: 27) |
| | Antisense: UUCUUCAGGAACCCAGGUG |
| | (SEQ ID NO: 28) |
| dsOct4-624 | Sense: AAGCACCUGGGUUCCUGAA |
| | (SEQ ID NO: 29) |
| | Antisense: UUCAGGAACCCAGGUGCUU |
| | (SEQ ID NO: 30) |
| dsOct4-626 | Sense: CAAGCACCUGGGUUCCUGA |
| | (SEQ ID NO: 31) |
| | Antisense: UCAGGAACCCAGGUGCUUG |
| | (SEQ ID NO: 32) |
| dsOct4-628 | Sense: GUCAAGCACCUGGGUUCCU |
| | (SEQ ID NO: 33) |
| | Antisense: AGGAACCCAGGUGCUUGAC |
| | (SEQ ID NO: 34) |
| dsOct4-631 | Sense: GGGGUCAAGCACCUGGGUU |
| | (SEQ ID NO: 35) |
| | Antisense: AACCCAGGUGCUUGACCCC |
| | (SEQ ID NO: 36) |
| dsOct4-636 | Sense: GAGAGGGGGUCAAGCACCU |
| | (SEQ ID NO: 37) |
| | Antisense: AGGUGCUUGACCCCCUCUC |
| | (SEQ ID NO: 38) |
| dsOct4-639 | Sense: GUGGAGAGGGGGUCAAGCA |
| | (SEQ ID NO: 39) |
| | Antisense: UGCUUGACCCCCUCUCCAC |
| | (SEQ ID NO: 40) |
| dsMYC-961 | Sense: GCA UAC AUA AUG CAU AAU A |
| | (SEQ ID NO: 41) |
| | Antisense: UAU UAU GCA UUA UGU AUG C |
| | (SEQ ID NO: 42) |
| dsMYC-826 | Sense: GAC ACA UCU CAG GGC UAA A |
| | (SEQ ID NO: 43) |
| | Antisense: UUU AGC CCU GAG AUG UGU C |
| | (SEQ ID NO: 44) |
| dsMYC-747 | Sense: UCU GCU GCU UUG GCA GCA A |
| | (SEQ ID NO: 45) |
| | Antisense: UUG CUG CCA AAG CAG CAG A |
| | (SEQ ID NO: 46) |
| dsMYC-690 | Sense: GAU AGC UGU GCA UAC AUA A |
| | (SEQ ID NO: 47) |
| | Antisense: UUA UGU AUG CAC AGC UAU C |
| | (SEQ ID NO: 48) |
| dsMYC-681 | Sense: GCA UAC AUA AUG CAU AAU A |
| | (SEQ ID NO: 49) |
| | Antisense: UAU UAU GCA UUA UGU AUG C |
| | (SEQ ID NO: 50) |
| dsMYC-496 | Sense: GCC CAU UAA UAC CCU UCU U |
| | (SEQ ID NO: 51) |
| | Antisense: AAG AAG GGU AUU AAU GGG C |
| | (SEQ ID NO: 52) |
| dsMYC-442 | Sense: GGC CCU UUC CCC AGC CUU A |
| | (SEQ ID NO: 53) |
| | Antisense: UAA GGC UGG GGA AAG GGC C |
| | (SEQ ID NO: 54) |
| dsMYC-316 | Sense: CAA GGA UGC GGU UUG UCA A |
| | (SEQ ID NO: 55) |
| | Antisense: UUG ACA AAC CGC AUC CUU G |
| | (SEQ ID NO: 56) |
| dsMYC-310 | Sense: UGC GGU UUG UCA AAC AGU A |
| | (SEQ ID NO: 57) |
| | Antisense: UAC UGU UUG ACA AAC CGC A |
| | (SEQ ID NO: 58) |
| dsMYC-261 | Sense: AGG GUU UGA GAG GGA GCA A |
| | (SEQ ID NO: 59) |
| | Antisense: UUG CUC CCU CUC AAA CCC U |
| | (SEQ ID NO: 60) |
| dsMYC-199 | Sense: ACU CUG UUU ACA UCC UAG A |
| | (SEQ ID NO: 61) |
| | Antisense: UCU AGG AUG UAA ACA GAG U |
| | (SEQ ID NO: 62) |
| dsNANOG-752 | Sense: GCCAGAUUUUGAGACACUA |
| | (SEQ ID NO: 63) |
| | Antisense: UAGUGUCUCAAAAUCUGGC |
| | (SEQ ID NO: 64) |
| dsNANOG-926 | Sense: GGGAUAGACAAGAAACCAA |
| | (SEQ ID NO: 65) |
| | Antisense: UUGGUUUCUUGUCUAUCCC |
| | (SEQ ID NO: 66) |
| dsKLF4-496 | Sense: GAA CCC AGG GAG CCG ACA A |
| | (SEQ ID NO: 67) |
| | Antisense: UUG UCG GCU CCC UGG GUU C |
| | (SEQ ID NO: 68) |

In certain embodiments, the method may comprise introducing into a somatic cell: (i) a saRNA molecule, wherein the saRNA molecule comprises a first ribonucleic acid strand comprising a ribonucleic acid sequence at least 80% complementary to a promoter region sequence of a first transcription factor gene, wherein the first transcription factor gene encodes a first transcription factor that induces the somatic cell to form a pluripotent stem cell, wherein the introducing is sufficient to activate transcription of the first transcription factor gene and (ii) a second saRNA molecule, wherein the saRNA molecule comprises a first ribonucleic acid strand comprising a ribonucleic acid sequence at least 80% complementary to a promoter region sequence of a second transcription factor gene, wherein the second transcription factor gene encodes a second transcription factor that induces the somatic cell to form a pluripotent stem cell, wherein the introducing is sufficient to activate transcription of the second transcription factor gene. In certain cases, the method may further comprise introducing into the somatic cell (iii) a third saRNA molecule, wherein the saRNA molecule comprises a first ribonucleic acid strand comprising a ribonucleic acid sequence at least 80% complementary to a promoter region sequence of a third transcription factor gene, wherein the transcription factor gene encodes a third transcription factor that induces the somatic cell to form a pluripotent stem cell, wherein the introducing is sufficient to activate transcription of the third transcription factor gene. In certain cases, the method may further comprise introducing into the somatic cell (iv) a fourth saRNA molecule, wherein the saRNA molecule comprises a first ribonucleic acid strand comprising a nucleic acid sequence at least 80% complementary to a promoter region sequence of a fourth transcription factor gene, wherein the transcription factor gene encodes a fourth transcription factor that induces the somatic cell to form a pluripotent stem cell, wherein the introducing is sufficient to activate transcription of the fourth transcription factor gene. In certain embodiments, the two or more saRNA molecules may be introduced into the somatic cell simultaneously. Two or more saRNA molecules targeting different transcription factors may be introduced into the somatic cell simultaneously by using a composition comprising the two or more saRNA molecules, for example. The first, second, third, and fourth transcription factors may be transcription factors described above as well as other transcription factors whose expression may induce a somatic cell to form an iPS cell. In certain cases, the first and second transcription factors may be a transcription factor of the Oct family, e.g., Oct4 and a transcription factor of the Sox family, e.g., Sox2. In certain cases, the third transcription factor may be a member of the Myc family, e.g., c-Myc. In certain cases, the third or fourth transcription factor may be a member of the: Myc family, e.g., c-My; Nanog family, e.g., Nanog; Lin family, e.g., Lin28; Klf family, e.g., Klf4; or NR5A family, e.g., NR5A2. Two or more saRNA molecules may be introduced into a somatic cell sequentially, simultaneously, or by a combination of sequential and simultaneous introduction.

In certain embodiments, the method may comprise introducing into a somatic cell: (i) a first saRNA molecule, wherein the saRNA molecule comprises a first ribonucleic acid strand comprising a ribonucleic acid sequence complementary to a promoter region sequence of a first transcription factor gene, wherein the first transcription factor gene encodes a first transcription factor that induces the somatic cell to form a pluripotent stem cell, wherein the introducing is sufficient to activate transcription of the first transcription factor gene and (ii) a nucleic acid encoding a second transcription factor that induces the somatic cell to form a pluripotent stem cell.

The nucleic acid encoding a second transcription factor may be operably linked to a constitutive or a conditional promoter. The nucleic acid can be delivered through a number of delivery systems such as, electroporation, nucleofection, lipofection, calcium phosphate precipitation, plasmids, viral vectors, viral nucleic acids, phage nucleic acids, phages, cosmids, or via carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art. Further, these methods can be used to target certain and cell populations by using the targeting characteristics of the carrier. In certain embodiments, the nucleic acid encoding a second transcription factor may be present in a vector. In certain cases, the vector may be a viral-based vector, such as a retroviral vector or a lentiviral vector.

In certain cases, the method may comprise introducing into a somatic cell: (i) a first saRNA molecule, wherein the saRNA molecule comprises a first ribonucleic acid strand comprising a ribonucleic acid sequence complementary to a promoter region sequence of a first transcription factor gene, wherein the first transcription factor gene encodes a first transcription factor that induces the somatic cell to form a pluripotent stem cell, wherein the introducing is sufficient to activate transcription of the first transcription factor gene, (ii) a nucleic acid encoding a second transcription factor that induces the somatic cell to form a pluripotent stem cell, and (iii) a nucleic acid encoding a third transcription factor that induces the somatic cell to form a pluripotent stem cell. In certain cases, the method may further comprise introducing into the somatic cell (iv) a nucleic acid encoding a fourth transcription factor that induces the somatic cell to form a pluripotent stem cell.

The saRNA molecule and one or more nucleic acids encoding transcription factors that induce the somatic cell to form a pluripotent stem cell may be introduced into the somatic cells sequentially, simultaneously, or by a combination of sequential and simultaneous introduction.

In certain embodiments, the transcription factor may be Oct4 and the first ribonucleic acid strand of the saRNA molecule may comprise a sequence complementary to a promoter region sequence of Oct 4 gene, wherein the sequence (GTCAAGCACCTGGGTTCCTGAAGAACATGGA (SEQ ID NO: 137)) is present between positions −628 to −598 relative to Oct4 transcription start site.

In certain embodiments, the transcription factor may be Oct4 and the first ribonucleic acid strand of the saRNA molecule may comprise a sequence selected from the group consisting of: SEQ ID NO: 1 to SEQ ID NO: 40 and SEQ ID NO: 69 to SEQ ID NO: 108. The saRNA molecule may comprise a second ribonucleic acid strand, wherein the second ribonucleic acid strand is complementary to the first ribonucleic acid strand. For example, when the first ribonucleic acid strand comprises a sequence from SEQ ID NO: 1, the second ribonucleic acid strand comprises a sequence from SEQ ID NO: 2 and when the first ribonucleic acid strand comprises a sequence from SEQ ID NO: 3, the second ribonucleic acid strand comprises a sequence from SEQ ID NO: 4. In certain embodiments, the first ribonucleic acid strand of the saRNA molecule may comprise a sequence selected from the group consisting of: SEQ ID NO: 21-SEQ ID NO:34 and SEQ ID NO: 89-102.

In certain embodiments, the transcription factor may be c-Myc and the first ribonucleic acid strand may comprise a sequence selected from SEQ ID NO: 41-SEQ ID NO: 62 and SEQ ID NO: 109-SEQ ID NO: 130. In certain embodiments, the first ribonucleic acid strand of the saRNA molecule may comprise a sequence selected from the group consisting of: SEQ ID NO: 47, SEQ ID NO: 48, SEQ ID NO: 55, and SEQ ID NO: 56.

The saRNA molecule may comprise a second ribonucleic acid strand, the first and second ribonucleic acid strands may be as shown in table 1.

In certain embodiments, the transcription factor may be Nanog and the first ribonucleic acid strand may comprise a sequence selected from SEQ ID NO: 63-SEQ ID NO: 66 and SEQ ID NO: 131-SEQ ID NO: 134. The saRNA molecule may comprise a second ribonucleic acid strand and when the first ribonucleic acid strand comprises a sequence from SEQ ID NO: 63, the second ribonucleic acid strand comprises a sequence from SEQ ID NO: 64 and when the first ribonucleic acid strand comprises a sequence from SEQ ID NO: 65, the second ribonucleic acid strand comprises a sequence from SEQ ID NO: 66.

In certain embodiments, the transcription factor may be Klf4 and the first ribonucleic acid strand may comprise a sequence selected from SEQ ID NO: 67, SEQ ID NO: 68, SEQ ID NO: 135, and SEQ ID NO: 136.

As described above, one or more saRNA molecule may be introduced into a somatic cell. In certain cases, the method for inducing a somatic cell to form an iPS cell may include introducing a plurality (two or more) saRNA molecules into the somatic cell, where the plurality of saRNA molecules have at least 80% complementarity to the promoter region of two or more transcription factors, as described above.

Compositions

As noted above the present disclosure provides saRNA molecules for use in inducing a somatic cell to form an iPS cell by activating transcription of a transcription factor that induces the somatic cell to form iPS cell.

A composition for activating transcription of a transcription factor that induces the somatic cell to form iPS cell may comprise a saRNA molecule, where the saRNA molecule is up to 30 nucleotides long and comprises at least one of a first ribonucleic acid strand comprising a ribonucleotide sequence at least 80% complementary to a promoter region sequence of a transcription factor that induces a somatic cell to form iPS cell.

In certain embodiments, the composition may comprise a saRNA molecule, where the saRNA molecule is up to 30 nucleotides long and comprises at least one of a first ribonucleic acid strand comprising a sequence at least 80% complementary to a sequence selected from SEQ ID NO: 1-SEQ ID NO: 136.

In certain embodiments, the composition may comprise a saRNA molecule comprising at least one of a first ribonucleic acid strand comprising a sequence at least 80% complementary to a sequence selected from: SEQ ID NO: 1 to SEQ ID NO: 40 and SEQ ID NO: 69 to SEQ ID NO: 108, wherein the sequence is sufficient to activate transcription of the Oct4 gene. In certain embodiments, the saRNA molecule may comprise a second ribonucleic acid strand at least 80% complementary to the first ribonucleic acid strand. For example, when the first ribonucleic acid strand comprises a sequence from SEQ ID NO: 1, the second ribonucleic acid strand comprises a sequence from SEQ ID NO: 2 and when the first ribonucleic acid strand comprises a sequence from SEQ ID NO: 3, the second ribonucleic acid strand comprises a sequence from SEQ ID NO: 4. In certain embodiments, the composition may comprise a second saRNA molecule comprising at least one ribonucleic acid strand, wherein when the first ribonucleic acid strand comprises a sequence from SEQ ID NO: 1, the second ribonucleic acid strand of the second saRNA molecule may comprise a sequence selected from SEQ ID NO: 3 or SEQ ID NO: 4. In certain embodiments, the first ribonucleic acid strand of the saRNA molecule may comprise a sequence selected from the group consisting of: SEQ ID NO: 21-SEQ ID NO:34 and SEQ ID NO: 89-102.

In certain embodiments, the composition may comprise a saRNA molecule comprising at least a first ribonucleic acid strand comprising a sequence selected from SEQ ID NO: 63-SEQ ID NO: 66 and SEQ ID NO: 131-SEQ ID NO: 134, wherein the sequence is complementary to a promoter region sequence of a Nanog gene and is sufficient to activate transcription of the Nanog gene. In certain embodiments, the composition may comprise a second ribonucleic acid strand and wherein when the first ribonucleic acid strand comprises a sequence from SEQ ID NO: 63, the second ribonucleic acid strand comprises a sequence from SEQ ID NO: 64 and when the first ribonucleic acid strand comprises a sequence from SEQ ID NO: 65, the second ribonucleic acid strand comprises a sequence from SEQ ID NO: 66.

In certain embodiments, the composition may comprise a second saRNA molecule comprising at least one ribonucleic acid strand, wherein when the first ribonucleic acid strand comprises a sequence from SEQ ID NO: 63, the one ribonucleic acid strand of the second saRNA comprises a sequence from SEQ ID NO: 65 or SEQ ID NO: 66.

In certain embodiments, the composition may comprise a saRNA molecule comprising at least a first ribonucleic acid strand comprising a sequence selected from SEQ ID NO: 41-SEQ ID NO: 62 and SEQ ID NO: 109-SEQ ID NO: 130, wherein the sequence is complementary to a promoter region sequence of a c-Myc gene and is sufficient to activate transcription of the c-Myc gene. In certain embodiments, the composition may comprise a second ribonucleic acid strand. Exemplary combination of first and second ribonucleic acid strands that may be present in a saRNA molecule are provided in tables 1 and 2.

In certain embodiments, the composition may comprise at least one saRNA molecule comprising a ribonucleic acid comprising a sequence having at least 99%, 95%, 90%, 85%, 80%, or less sequence identity to a sequence selected from the sequence of SEQ ID NO: 1-136.

In certain embodiments, the composition may comprise two or more saRNA molecules, where the ribonucleic acid strand(s) of each saRNA is complementary to a different sequence within the promoter region of the target gene (e.g., c-Myc gene). In certain embodiments, the composition may comprise two or more saRNA molecules, where the ribonucleic acid strand(s) of each saRNA is complementary to different sequences within the promoter regions of two or more target genes (e.g., Oct4 gene and KLF4 gene).

The saRNA molecules may comprise moieties other than ribonucleotide moieties, including, but not limited to, modified nucleotides, modified internucleotide linkages, non-nucleotides, deoxynucleotides and analogs of the aforementioned nucleotides.

saRNAs compounds of the present invention can be duplexes, and can be composed of separate strands or can comprise of a single strand of RNA that forms short hairpin dsRNAs with a hairpin loop as long as, for example, about 3 to about 23 or more nucleotides, about 5 to about 22, about 6 to about 21, about 7 to about 20, about 8 to about 19, about 9 to about 18, about 10 to about 17, about 11 to about 16, about 12 to about 15, about 13 to about 14 nucleotides, such as 3, or 4, or 5, or 7, or 12, or 18, or 21 nucleotides. RNAs having loops or hairpin loops can include structures where the loops are connected to the stem by linkers such as flexible linkers. Flexible linkers can be selected of a wide variety of chemical structures, as long as they are of sufficient length and materials to enable effective intramolecular hybridization of the stem elements.

Although the sequences in the SEQ ID NOs disclosed herein are perfectly complementary to a region in the promoter sequence of the target gene, in some embodiments, the ribonucleotide strand may comprise a sequence that is less than 100% complementary to a sequence in the promoter region of the target gene, including about 99% complementary, 98% complementary, 97% complementary, 96% complementary, 95% complementary, 94% complementary, 93% complementary, 92% complementary, 91% complementary, 90% complementary, 85% complementary, 80% complementary, 75% complementary, 70% complementary to the promoter region of the target gene.

The nucleotides of the saRNA, or at least one strand of a duplex saRNA, may be modified so as to provide a desired characteristic. For example, the saRNA molecules of the invention can comprise modification of a naturally occurring or non-naturally occurring polynucleotide that provides for enhanced nuclear uptake. An example of a nuclear uptake enhancing modification is a stabilizing modification, such as a modified internucleotide linkage, that confers sufficient stability on a molecule, such as a nucleic acid, to render it sufficiently resistant to degradation (e.g., by nucleases) such that the associated nucleic acid can accumulate in the nucleus of a cell when exogenously introduced into the cell. In this example, entry into the cell's nucleus is facilitated by the ability of the modified nucleic acid to resist nucleases sufficiently well such that an effective concentration of the nucleic acid can be achieved inside the nucleus.

Furthermore, the saRNA can be 2′-O-bis(2-hydroxyethoxy)methyl orthoester modified to provide for stability of the ribonucleic acid molecule. Other modification, include, for example a backbone phosphate group modification (e.g., methylphosphonate, phosphorothioate, phosphoroamidate and phosphorodithioate internucleotide linkages), which modifications can, for example, enhance their stability in vivo, making them particularly useful in therapeutic applications. A particularly useful phosphate group modification is the conversion to the phosphorothioate or phosphorodithioate forms of the saRNA. Phosphorothioates and phosphorodithioates are more resistant to degradation in vivo than their unmodified oligonucleotide counterparts, increasing the half-lives of the saRNA making them more available to the subject being treated. A saRNA may also be modified to comprise N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A number of nucleotide modifications as well as backbone phosphate group modifications may be utilized to increase the stability of saRNA. Such modifications are described in U.S. Patent Application Ser. No. 61/329,057, filed on Apr. 28, 2010; U.S. Application Publication No. 20050080246, U.S. Application Publication No. 20070032441, U.S. Pat. No. 7,683,036, which are hereby incorporated by reference.

The saRNA may be synthesized by any method that is now known or that comes to be known for synthesizing saRNA molecules and that from reading this disclosure, one skilled in the art would conclude would be useful in connection with the present invention. For example, one may use methods of chemical synthesis such as methods that employ Dharmacon, Inc.'s proprietary ACE® technology. Alternatively, one could also use template dependant synthesis methods. Synthesis may be carried out using modified or non-modified, natural or non-natural bases as disclosed herein. Moreover, synthesis may be carried out with or without modified or non-modified nucleic acid backbone as disclosed herein.

In addition, the saRNA molecules may be synthesized in a host cell by any method that is now known or that comes to be known for synthesizing saRNA molecules in a host cell. For example, saRNA molecules can be expressed from recombinant circular or linear DNA vector using any suitable promoter. Suitable promoters for expressing saRNA molecules of the invention from a vector include, for example, the U6 or H1 RNA pol III promoter sequences and the cytomegalovirus promoter. Selection of other suitable promoters is within the skill in the art. Suitable vectors for use with the subject invention include those described in U.S. Pat. No. 5,624,803, the disclosure of which is incorporated herein in its entirely. The recombinant plasmids of the invention can also comprise inducible or regulatable promoters for expression of the saRNA molecule in a particular tissue or in a particular intracellular environment.

The saRNA molecules of the invention can be expressed from a recombinant nucleic acid vector either as two separate, complementary RNA molecules, or as a single RNA molecule with two complementary regions. Selection of vectors suitable for expressing saRNA of the invention, methods for inserting nucleic acid sequences for expressing the saRNA into the vector, and methods of delivering the recombinant vector to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference. Other methods for delivery and intracellular expression suitable for use in the invention are described in, for example, U.S. Patent Application Publication Nos. 20040005593, 20050048647, 20050060771, the entire disclosures of which are herein incorporated by reference.

Once synthesized, the polynucleotides of the present invention may immediately be used or be stored for future use. In some embodiments, the polynucleotides of the invention are stored as duplexes in a suitable buffer. Many buffers are known in the art suitable for storing saRNAs. For example, the buffer may be comprised of 100 mM KCl, 30 mM HEPES-pH 7.5, and 1 mM MgCl₂. In representative embodiments, the double stranded polynucleotides of the present invention retain 30% to 100% of their activity when stored in such a buffer at 4° C. for one year. More preferably, they retain 80% to 100% of their biological activity when stored in such a buffer at 4° C. for one year. Alternatively, the compositions can be stored at −20° C. in such a buffer for at least a year or more. Usually, storage for a year or more at −20° C. results in less than a 50% decrease in biological activity. More usually, storage for a year or more at −20° C. results in less than a 20% decrease in biological activity after a year or more. Furthermore, storage for a year or more at −20° C. results in less than a 10% decrease in biological activity.

In order to ensure stability of the saRNA prior to usage, they may be retained in dry form (e.g., lyophilized form) at −20° C. until they are ready for use. Prior to usage, they should be resuspended; however, even once resuspended, for example, in the aforementioned buffer, they should be kept at −20° C. until used. The aforementioned buffer, prior to use, may be stored at approximately 4° C. or room temperature. Effective temperatures at which to conduct transfection are well known to persons skilled in the art, but include for example, room temperature.

### iPS Cells

The present disclosure provides an iPS cell comprising at least one exogenous saRNA molecule as described above. For example, the iPS cell may include an saRNA molecule comprising a first ribonucleic acid strand comprising a ribonucleic acid sequence at least 80% complementary to a promoter region sequence of a transcription factor gene, wherein the transcription factor gene encodes a transcription factor that induces a somatic cell to form a pluripotent stem cell. In certain embodiments, the saRNA molecule may further comprise a second ribonucleic acid strand comprising a ribonucleic acid sequence complementary to the ribonucleic acid of the first acid strand.

As described in the foregoing sections, the transcription factor gene may be Oct4, Sox2, c-Myc, Klf4, Nanog, Lin28 or NR5A2.

The iPS cell may possess two or more, or three or more, or four or more, or five or more, or six or more, or more, for example, seven properties including but not limited to expression of particular proteins, an ES cell like morphology, pluripotency, growth properties, epigenetic reprogramming, as described above.

The iPS cell may be made by the methods described herein. For example, the iPS cell may be made by introducing into a somatic cell a saRNA molecule, wherein the saRNA molecule comprises a first ribonucleic acid strand comprising a ribonucleic acid sequence at least 80% complementary to a promoter region sequence of a transcription factor gene, wherein the transcription factor gene encodes a transcription factor that induces the somatic cell to form a pluripotent stem cell, wherein the introducing is sufficient to activate the transcription of the transcription factor gene; and culturing the somatic cell to produce an induced pluripotent stem cell.

### Kits

Kits with the subject saRNA molecules and composition s comprising the saRNA molecules are provided. The kit may comprise one or more containers in which the subject saRNA molecules may be present in a dried (e.g., lyophilized) form or a solution form. The subject saRNA molecules may be provided in separate containers or a combination of certain number of saRNA molecules may be provided in a single container.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The sequences of saRNA molecules used in the experiments described herein are provided in Table 2 below.

TABLE 2
Sequences for saRNAs

| saRNA name | Sequence (5′-3′) |
|---|---|
| dsOct4-141 | Sense: GCCACCACCAUUAGGCAAA[dT][dT] |
| | (SEQ ID NO: 69) |
| | Antisense: UUUGCCUAAUGGUGGUGGC[dT][dT] |
| | (SEQ ID NO: 70) |
| dsOct4-162 | Sense: UCCCCUUCCACAGACACCA[dT][dT] |
| | (SEQ ID NO: 71) |
| | Antisense: UGGUGUCUGUGGAAGGGGA[dT][dT] |
| | (SEQ ID NO: 72) |
| dsOct4-257 | Sense: AGU GAG ACC CUG UCU UAA A[dT][dT] |
| | (SEQ ID NO: 73) |
| | Antisense: UUU AAG ACA GGG UCU CAC U[dT] |
| | [dT] (SEQ ID NO: 74) |
| dsOct4-275 | Sense: CUCCAGUCUGGGCAACAAA[dT][dT] |
| | (SEQ ID NO: 75) |
| | Antisense: UUUGUUGCCCAGACUGGAG[dT][dT] |
| | (SEQ ID NO: 76) |
| dsOct4-420 | Sense: GGCCCCAUCUCUACUAAAA[dT][dT] |
| | (SEQ ID NO: 77) |
| | Antisense: UUUUAGUAGAGAUGGGGCC[dT][dT] |
| | (SEQ ID NO: 78) |
| dsOct4-422 | Sense: AAG GCC CCA UCU CUA CUA A[dT][dT] |
| | (SEQ ID NO: 79) |
| | Antisense: UUA GUA GAG AUG GGG CCU U[dT] |
| | [dT] (SEQ ID NO: 80) |
| dsOct4-573 | Sense: UGGGAUGUGCAGAGCCUGA[dT][dT] |
| | (SEQ ID NO: 81) |
| | Antisense: UCAGGCUCUGCACAUCCCA[dT][dT] |
| | (SEQ ID NO: 82) |
| dsOct4-581 | Sense: CAGACAGCUGGGAUGUGCA[dT][dT] |
| | (SEQ ID NO: 83) |
| | Antisense: UGCACAUCCCAGCUGUCUG[dT][dT] |
| | (SEQ ID NO: 84) |
| dsOct4-586 | Sense: GAUUCCAGACAGCUGGGAU[dT][dT] |
| | (SEQ ID NO: 85) |
| | Antisense: AUCCCAGCUGUCUGGAAUC[dT][dT] |
| | (SEQ ID NO: 86) |
| dsOct4-594 | Sense: GUGGGAGUGAUUCCAGACA |
| | (SEQ ID NO: 87) |
| | Antisense: UGUCUGGAAUCACUCCCAC[dT][dT] |
| | (SEQ ID NO: 88) |
| dsOct4-598 | Sense: AGGUGUGGGAGUGAUUCCA[dT][dT] |
| | (SEQ ID NO: 89) |
| | Antisense: UGGAAUCACUCCCACACCU[dT][dT] |
| | (SEQ ID NO: 90) |
| dsOct4-616 | Sense: GGUUCCUGAAGAACAUGGA[dT][dT] |
| | (SEQ ID NO: 91) |
| | Antisense: UCCAUGUUCUUCAGGAACC[dT][dT] |
| | (SEQ ID NO: 92) |
| dsOct4-620 | Sense: CCUGGGUUCCUGAAGAACA[dT][dT] |
| | (SEQ ID NO: 93) |
| | Antisense: UGUUCUUCAGGAACCCAGG[dT][dT] |
| | (SEQ ID NO: 94) |
| dsOct4-622 | Sense: CACCUGGGUUCCUGAAGAA[dT][dT] |
| | (SEQ ID NO: 95) |
| | Antisense: UUCUUCAGGAACCCAGGUG[dT][dT] |
| | (SEQ ID NO: 96) |
| dsOct4-624 | Sense: AAGCACCUGGGUUCCUGAA[dT][dT] |
| | (SEQ ID NO: 97) |
| | Antisense: UUCAGGAACCCAGGUGCUU[dT][dT] |
| | (SEQ ID NO: 98) |
| dsOct4-626 | Sense: CAAGCACCUGGGUUCCUGA[dT][dT] |
| | (SEQ ID NO: 99) |
| | Antisense: UCAGGAACCCAGGUGCUUG[dT][dT] |
| | (SEQ ID NO: 100) |
| dsOct4-628 | Sense: GUCAAGCACCUGGGUUCCU[dT][dT] |
| | (SEQ ID NO: 101) |
| | Antisense: AGGAACCCAGGUGCUUGAC[dT][dT] |
| | (SEQ ID NO: 102) |
| dsOct4-631 | Sense: GGGGUCAAGCACCUGGGUU[dT][dT] |
| | (SEQ ID NO: 103) |
| | Antisense: AACCCAGGUGCUUGACCCC |
| | (SEQ ID NO: 104) |
| dsOct4-636 | Sense: GAGAGGGGGUCAAGCACCU[dT][dT] |
| | (SEQ ID NO: 105) |
| | Antisense: AGGUGCUUGACCCCCUCUC[dT][dT] |
| | (SEQ ID NO: 106) |
| dsOct4-639 | Sense: GUGGAGAGGGGGUCAAGCA[dT][dT] |
| | (SEQ ID NO: 107) |
| | Antisense: UGCUUGACCCCCUCUCCAC[dT][dT] |
| | (SEQ ID NO: 108) |
| dsMYC-961 | Sense: GCA UAC AUA AUG CAU AAU A[dT][dT] |
| | (SEQ ID NO: 109) |
| | Antisense: UAU UAU GCA UUA UGU AUG C[dT] |
| | [dT] (SEQ ID NO: 110) |
| dsMYC-826 | Sense: GAC ACA UCU CAG GGC UAA A[dT][dT] |
| | (SEQ ID NO: 111) |
| | Antisense: UUU AGC CCU GAG AUG UGU C[dT] |
| | [dT] (SEQ ID NO: 112) |
| dsMYC-747 | Sense: UCU GCU GCU UUG GCA GCA A[dT][dT] |
| | (SEQ ID NO: 113) |
| | Antisense: UUG CUG CCA AAG CAG CAG A[dT] |
| | [dT] (SEQ ID NO: 114) |
| dsMYC-690 | Sense: GAU AGC UGU GCA UAC AUA A[dT][dT] |
| | (SEQ ID NO: 115) |
| | Antisense: UUA UGU AUG CAC AGC UAU C[dT] |
| | [dT] (SEQ ID NO: 116) |
| dsMYC-681 | Sense: GCA UAC AUA AUG CAU AAU A[dT][dT] |
| | (SEQ ID NO: 117) |
| | Antisense: UAU UAU GCA UUA UGU AUG C[dT] |
| | [dT] (SEQ ID NO: 118) |
| dsMYC-496 | Sense: GCC CAU UAA UAC CCU UCU U[dT][dT] |
| | (SEQ ID NO: 119) |
| | Antisense: AAG AAG GGU AUU AAU GGG C[dT] |
| | [dT] (SEQ ID NO: 120) |
| dsMYC-442 | Sense: GGC CCU UUC CCC AGC CUU A[dT][dT] |
| | (SEQ ID NO: 121) |
| | Antisense: UAA GGC UGG GGA AAG GGC C[dT] |
| | [dT] (SEQ ID NO: 122) |
| dsMYC-316 | Sense: CAA GGA UGC GGU UUG UCA A[dT][dT] |
| | (SEQ ID NO: 123) |
| | Antisense: UUG ACA AAC CGC AUC CUU G[dT] |
| | [dT] (SEQ ID NO: 124) |
| dsMYC-310 | Sense: UGC GGU UUG UCA AAC AGU A[dT][dT] |
| | (SEQ ID NO: 125) |
| | Antisense: UAC UGU UUG ACA AAC CGC A[dT] |
| | [dT] (SEQ ID NO: 126) |
| dsMYC-261 | Sense: AGG GUU UGA GAG GGA GCA A[dT][dT] |
| | (SEQ ID NO: 127) |
| | Antisense: UUG CUC CCU CUC AAA CCC U[dT] |
| | [dT] (SEQ ID NO: 128) |
| dsMYC-199 | Sense: ACU CUG UUU ACA UCC UAG A[dT][dT] |
| | (SEQ ID NO: 129) |
| | Antisense: UCU AGG AUG UAA ACA GAG U[dT] |
| | [dT] (SEQ ID NO: 130) |
| dsNANOG-752 | Sense: GCCAGAUUUUGAGACACUA[dT][dT] |
| | (SEQ ID NO: 131) |
| | Antisense: UAGUGUCUCAAAAUCUGGC[dT][dT] |
| | (SEQ ID NO: 132) |
| dsNANOG-926 | Sense: GGGAUAGACAAGAAACCAA[dT][dT] |
| | (SEQ ID NO: 133) |
| | Antisense: UUGGUUUCUUGUCUAUCCC[dT][dT] |
| | (SEQ ID NO: 134) |
| dsKLF4-496 | Sense: GAA CCC AGG GAG CCG ACA A[dT][dT] |
| | (SEQ ID NO: 135) |
| | Antisense: UUG UCG GCU CCC UGG GUU C[dT] |
| | [dT] (SEQ ID NO: 136) |

### Example 1

### saRNA Induce Potent and Prolonged Transcriptional Gene Activation

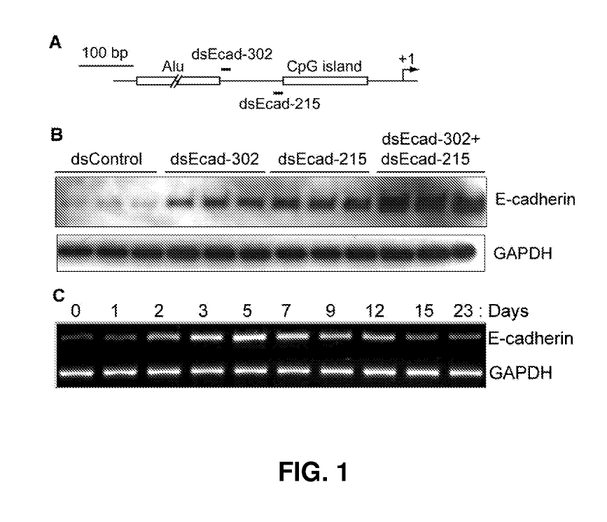

saRNA molecules that target the promoter sequences of genes can induce sequence-specific transcriptional activation, a phenomenon referred to as RNAa. RNAa is potent and long-lasting (FIG. 1).

Two saRNAs were designed to target the E-cadherin promoter at location −302 and −215 relative to the transcription start site (TSS) (FIG. 1, panel A). The saRNA were transfected alone or in combination into human prostate cancer cells (PC-3) at a concentration of 50 nM. E-cadherin protein expression was evaluated by Western blotting analysis. Individual saRNA caused robust activation of E-cadherin protein expression and had a greater effect when combined (FIG. 1, panel B). A time-course experiment showed the prolonged activation of E-cadherin mRNA expression after a single transfection of 50 nM dsEcad-215 (FIG. 1, panel C).

These properties suggest RNAa is well suited for reprogramming stem cell genes.

### Example 2

### Promoter-Targeted saRNAs Activate Oct4 Expression in Somatic Cells

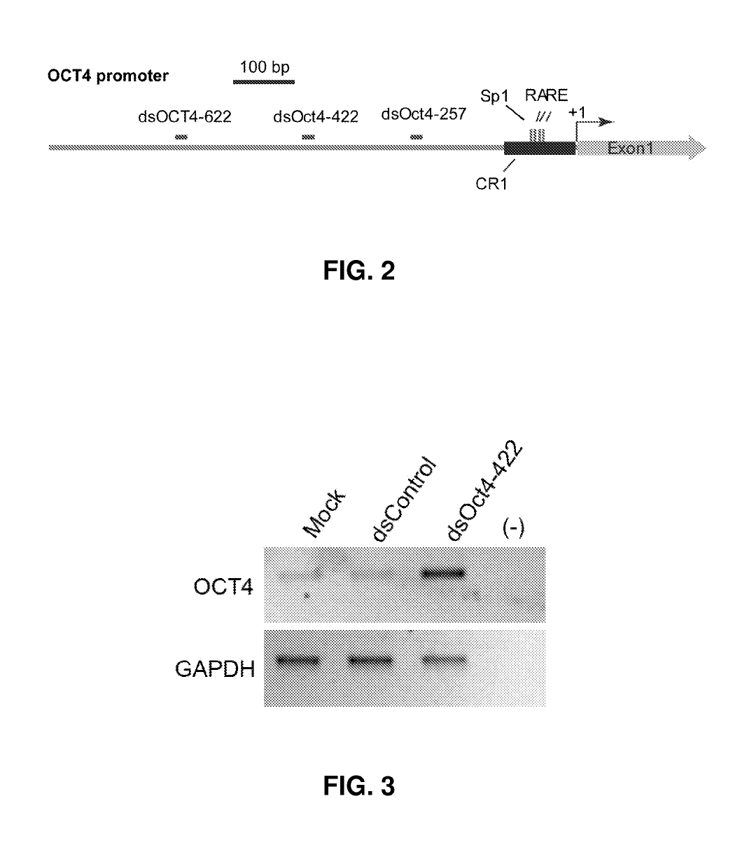

To test whether the stem cell transcription factor Oct4 can be activated by RNAa, several saRNAs to target the Oct4 promoter were designed (FIG. 2). One-kb of promoter sequences (upstream of the TSS) for the human Oct4 gene were retrieved from the Ensembl genome database. Three saRNA targets were selected on the Oct4 promoter (FIG. 2) based on the rules previously described (Li L C, et al., Proceedings of the National Academy of Sciences of the United States of America 2006; 103:17337-42 and Huang V, et al., PLoS One. 2010 Jan. 22;5(1):e8848). The saRNAs were chemically synthesized and transfected into HeLa cells, a cancer cell line known to express Oct4. As shown in FIG. 3, Oct4 mRNA expression was dramatically increased in dsOct4-422 transfected cells compared to mock or control saRNA-transfected cells.

The Oct4 promoter is schematically represented in FIG. 2. The transcription start site is designated as +1. A conserved region (CR1) between human and mouse, an Spl binding site, and three retinoic acid responsive elements (RAREs) are indicated. Three saRNAs targeting locations −257, −422 and −622 relative to the transcription start site are shown.

saRNAs targeting location −422 of the human OCT gene promoter were transfected into HeLa cells at a concentration of 50 nM using Lipofectamine 2000 (Invitrogen). RNA was isolated from transfected cells and treated with DNA-free DNase (Ambion) to eliminate potential DNA contamination. The resulting RNA (1 μg) was reverse transcribed into cDNA using oligo(dT) primers. The resulting cDNA was amplified using Oct4- and GAPDH-specific primers. The Oct4 primers were carefully designed to exclude any amplification from Oct4 pseudogenes; their sequences are Oct4-Sense: TCCCTTCGCAAGCCCTCAT (SEQ ID NO: 138) and Oct4-Antisense: TGACGGTGCAGGGCTCCGGGGAGGCCCCATC (SEQ ID NO: 139).

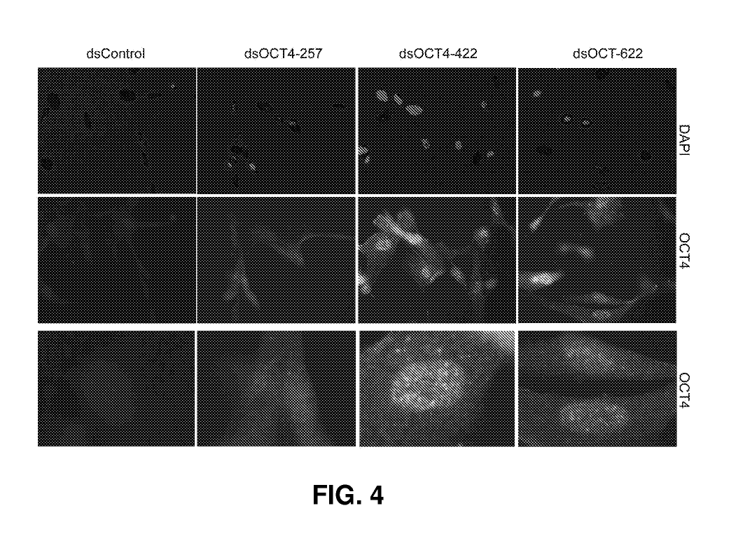

Activation of Oct4 expression in primary cells that do not express Oct4 was tested. IMR-90 human fibroblast cell line was chosen for this experiment because it was shown to be transformed into iPS cells following lentiviral transduction of four transcription factors (Yu J et al., Human Somatic Cells, Science, 2007, Dec. 21; 318 (5858):1917-20). IMR-90 cells were obtained from ATCC(CCL-186). saRNA was transfected into IMR-90 cells using the Nucleofector device and the Cell Line Nucleofector Kit R (Amaxa). Seventy-two hours later, the cells were fixed and stained for Oct4 protein expression. As shown in FIG. 4, no Oct4 staining was detected in the cells transfected with control saRNA. However, Oct4 expression was dramatically induced by dsOct4-422 or dsOct4-622, as evidenced by the punctate staining of Oct4 located in the nucleus. dsOCT-257 had no apparent effect on Oct4 expression.

IMR-90 cells were cultured on coverslips and nucleofected with 100 nM of control saRNA or Oct4 saRNA. Cells were fixed 72 hrs later with 4% paraform-aldehyde and permeablized with 0.2% Triton X-100. After blocking for 60 min with 10% normal goat serum, cells were incubated with anti-Oct4 antibody (Abcam) overnight at 4° C. The cells were washed with PBS and incubated for 2 hrs with FITC-conjugated secondary antibodies. For reference, nuclei were stained with DAPI for 5 min before final embedding. Fluorescent images were obtained using a Nikon E600 microscope equipped with a digital camera. FIG. 4, top row, DAPI nuclear staining; FIG. 4, middle row, Oct4 staining; FIG. 4, bottom row, regional magnified view of the corresponding images in the middle row.

### Example 3

High-Throughput Screening for Stem Cell Transcription Factor Specific saRNA

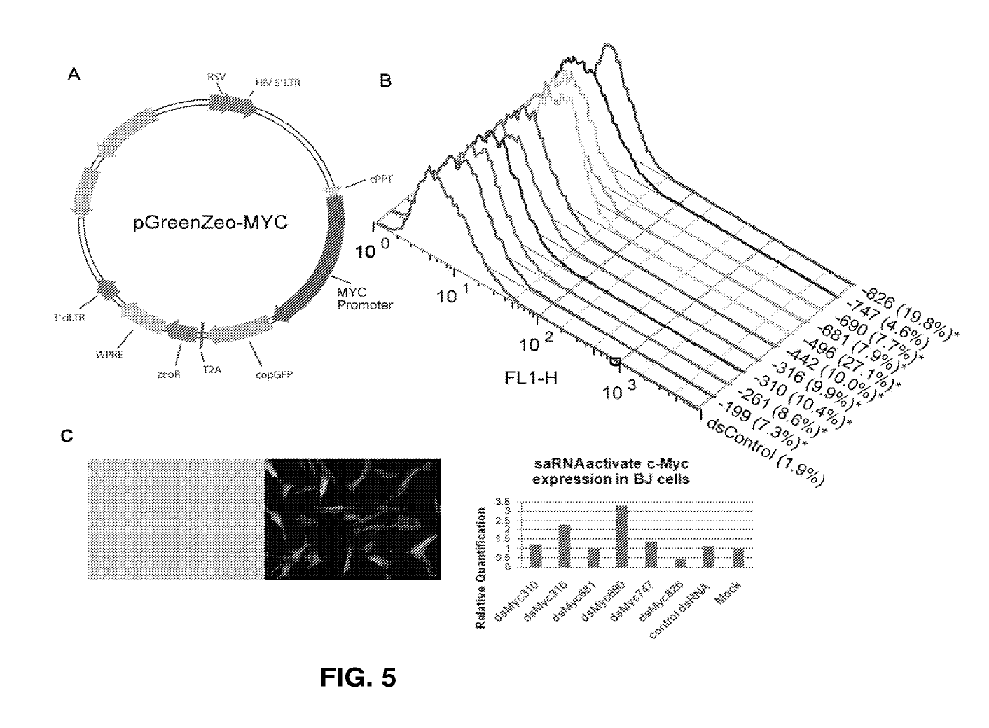

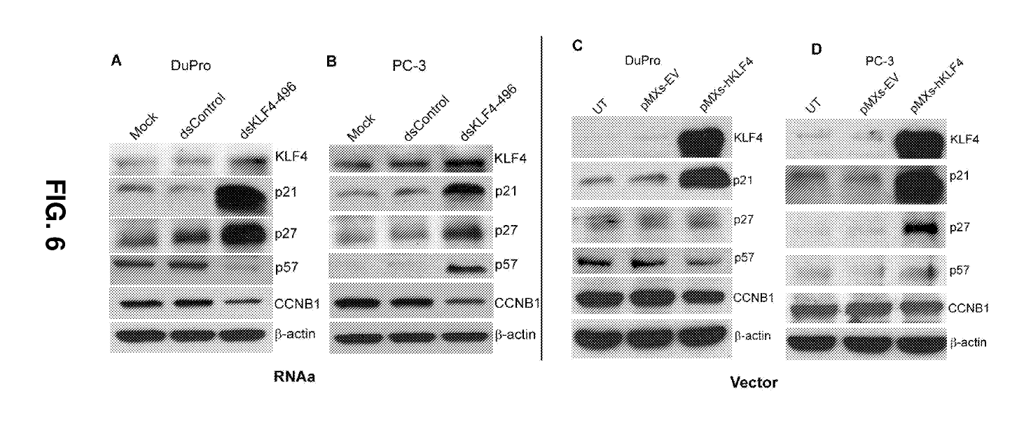

Genome-integrated lentiviral reporter gene system allows for high-throughput readout of transcriptional activity by FACS analysis of GFP expression while providing promoter environments similar to that for endogenous promoters. To demonstrate the feasibility of this system for saRNA screening, a GFP based lentiviral reporter system (pGreenZero or pGZ) was adapted. Lentiviral expression vectors are the most effective vehicles for delivering genetic material to those difficult-to-transfect cells, like BJ cells, a human primary fibroblast cell line. The promoter sequence for Oct4, NANOG, SOX2 and c-Myc were cloned by PCR and inserted into pGZ to drive GFP expression. The cloned promoter sequences for these genes range from 2 kb to 5.1 kb (FIG. 5, panel A). The resulting constructs were packaged and used to infect BJ cells. CMV promoter driven GFP expression was used as positive control. More than 90% BJ cells infected by pGZCMV were GFP positive suggesting high infection efficiency (FIG. 5, panel C). pGZMyc transducted BJ cells were then nucleofected with c-Myc saRNA. GFP-positive cells were analyzed by flow cytometry (FIG. 5, panel B). A significant increase in GFP-positive population was observed in most c-Myc promoter-targeted saRNA transfected BJ cells compared to the control saRNA transfected cells (FIG. 5, panel B). To further confirm the effects of these saRNAs, mRNA expression was analyzed by quantitative RT-PCR (FIG. 5, panel D). Consistent with the flow cytometry results, an increased mRNA level was observed in BJ cells nucleofected by dsMyc-316 and dsMyc-690.

FIG. 5, panel A. Vector map of the lentiviral pGreenZeo-MYC reporter in which a 5.1 kb human MYC promoter was inserted upstream of the GFP gene. FIG. 5, panel B. BJ cells were infected with pGreenZeo-MYC lentiviral particles and then nucleofected with a control or c-Myc saRNAs. Cells were harvested for FACS analysis of GFP expression 72 hrs later. FACS data were analyzed using the FlowJo program. The numbers [−nnn (n.n %)] on the right of the figure indicate target location relative to the TSS and the percentage of GFP-positive cells. *, Significant difference in % of GFP-positive cells compared to dsControl (χ²>4). FIG. 5, panel C. As a control for infection efficiency, lentivirus particles generated from a CMV promoter driven GFP expression vector was used to infect BJ cells. More than 90% of the cells are infected. FIG. 5, panel D. c-Myc mRNA expression levels in BJ cells after saRNA nucleofection as assessed by qRT-PCR. The value is relative to beta actin. Mock control of BJ cells was set to 1.

### Example 5

### Functional Evaluation of saRNA-Mediated Overexpression of Stem Cell Transcription Factors

To evaluate whether and to what extent saRNA-mediated overexpression of endogenous genes restores their natural function as transcription factors, saRNA mediated activation of KLF4, one of the stem cell transcription factors in Yamanaka's reprogramming recipe (Takahashi K, et al., Cell 2007; 131:861-72) was tested. KLF4 was activated using a saRNA in several prostate cancer cell lines. The expression of known KLF4 regulated downstream genes was assessed. The expression was compared to that induced by retroviral vector based KLF4 overexpression. Through screening several KLF4 promoter-targeted saRNAs in cancer cell lines, dsKLF4-496 was identified as a saRNA that robustly activates KLF4 expression in several prostate cancer cell lines (FIG. 6, panels A and B). KLF4 is known to regulate the expression of several cell cycle-related genes including p21^{WAF1/CIP1} (p21), p27^{KIP1} (p27), p57^{KIP}2 (p⁵⁷), and Cyclin B1 CCNB1). To determine if RNAa-based overexpression of KLF4 modulated the expression of downstream cell cycle genes, protein levels of p21, p27, p57 and CCNB1 were evaluated in DuPro and PC-3 cells following dsKLF4-496 transfection. As shown in FIG. 6, panels A and B, dsKLF4-496 transfection altered the expression of several downstream targets. To validate the results obtained through saRNA-mediated activation of KLF4, a retroviral transduction system was utilized to overexpress KLF4 cDNA in these two cell lines. Infection of KLF4 viral particles caused robust induction of KLF4 protein levels and modulation of KLF4 downstream target genes in a pattern similar to saRNA mediated overexpression (FIG. 6, panel C and D). These results thus validate saRNA as a tool for restoring endogenous gene function. Interestingly, saRNA appeared to facilitate a greater measurable effect on downstream gene expression compared to viral transduction despite the magnitude of KLF4 induction by saRNA being less than vector based overexpression (FIG. 6). It is likely that saRNA has restored a more natural and potent function of KLF4 as a transcription factor via simultaneous activation of additional KLF4 splicing variants.

FIG. 6, panels A and B. DuPro and PC-3 cells were mock transfected or transfected with 50 nM of the indicated saRNA. The transfected cells were harvested at 96 hrs following transfection for protein expression analysis by Western blotting assay. FIG. 6, panels C and D. DuPro and PC-3 cells were untreated (UT) or infected with retroviral particles expressing an empty vector (pMXs-EV) or a vector coding human KLF4 cDNA (pMXs-hKLF4). Protein levels of genes were analyzed at 96 hrs following infection by Western blotting using protein specific antibodies.

### Example 6

### NANOG Gene Activation by saRNA

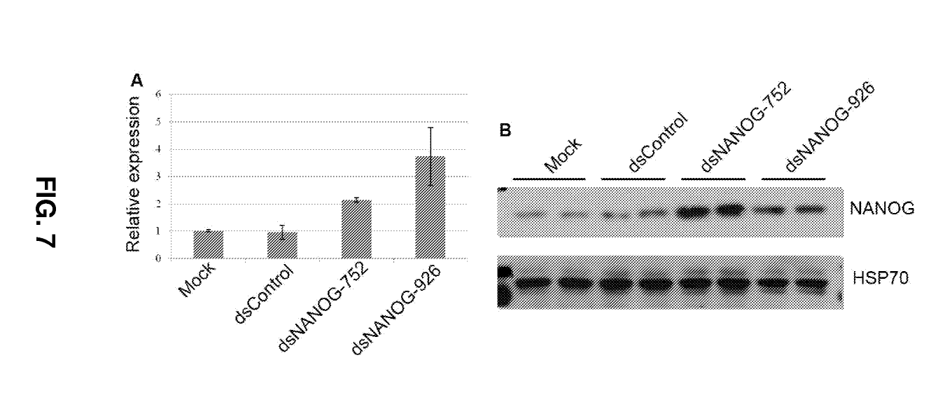

NANOG is one of the four stem cell factors used by Yu et al. to reprogram human iPS cells (Yu J et al., supra). Two saRNA on NANOG promoter at the location of −752 and −926 relative to the transcription start site were designed. These saRNA were transfected into a human embryonic cancer cell line NCCIT. Subsequent expression analysis of the transfected cells revealed that NANOG was activated by both saRNAs by 2- to 3.7-fold at the mRNA level (FIG. 7, panel A). In consistence, NANOG protein expression was also induced by both saRNAs (FIG. 7, panel B) NCCIT cells were transfected with 50 nM of the indicated saRNA for 72 hrs. NANOG expression at the mRNA level (FIG. 7, panel A) and protein level (FIG. 7, panel B) was assessed by real-time RT-PCR and Western blotting analysis, respectively.

### Example 7

### Replacement of Viral Vector by saRNA Enhances iPS Reprogramming Efficiency

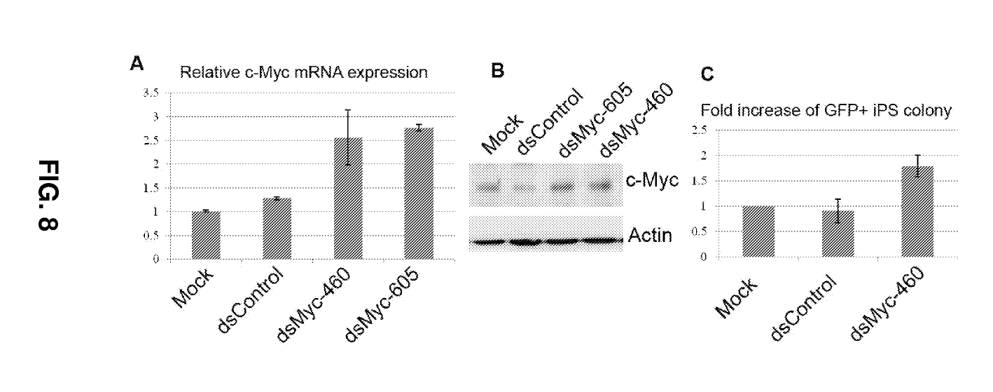

To demonstrate reprogramming of somatic cells, two saRNAs (dsMyc-460 and -605) that target mouse c-Myc promoter were identified from a screen. Transfection of these two saRNA caused a >2.5-fold induction of c-Myc mRNA expression in mouse embryonic fibroblasts (MEFs) isolated from OG2 mice that transgenically express green fluorescence protein (GFP) driven by Oct4 promoter (Oct4-GFP) (FIG. 8, panel A). c-Myc protein expression was also induced by these two saRNAs (FIG. 8, panel B). The c-Myc virus was replaced with dsMyc-460 in a four factor reprogramming system that includes Oct4, Sox2, c-Myc and Klf4. As shown in FIG. 8, panel C, dsMyc-460 resulted in 1.75-fold increase in the number of colonies compared to mock and control saRNA in reprogramming OG2-MEF cells in combination with the remaining 3 factors (Oct4, Sox2 and Klf4). This data thus provide the first evidence that saRNA could be used alone or in combination with virus to reprogram somatic cells into iPS cells.

FIG. 8, panel A. Mouse MEF cells were transfected with the indicated saRNA for 72 hrs. mRNA expression of c-Myc was analyzed by real-time RT-PCR in the transfected cells. Results are means±SD of two independent experiments. FIG. 8, panel B. Mouse MEF cells were transfected as in FIG. 8, panel A. c-Myc protein levels were detected by Western blotting analysis. FIG. 8, panel C, OG2-MEF cells were infected with virus particles that expressed Oct4, Sox2 and KLF4, and transfected with 50 nM of the indicated saRNA or mock transfected. After 72 hrs, the cells were cultured in ES cell medium. GFP positive colonies were counted at day 11 following infection and transfection. Results are means±SD of two independent experiments and plotted as fold increase relative to mock treatment.

### Example 8

### Screen for Oct4-Specific saRNA Molecules

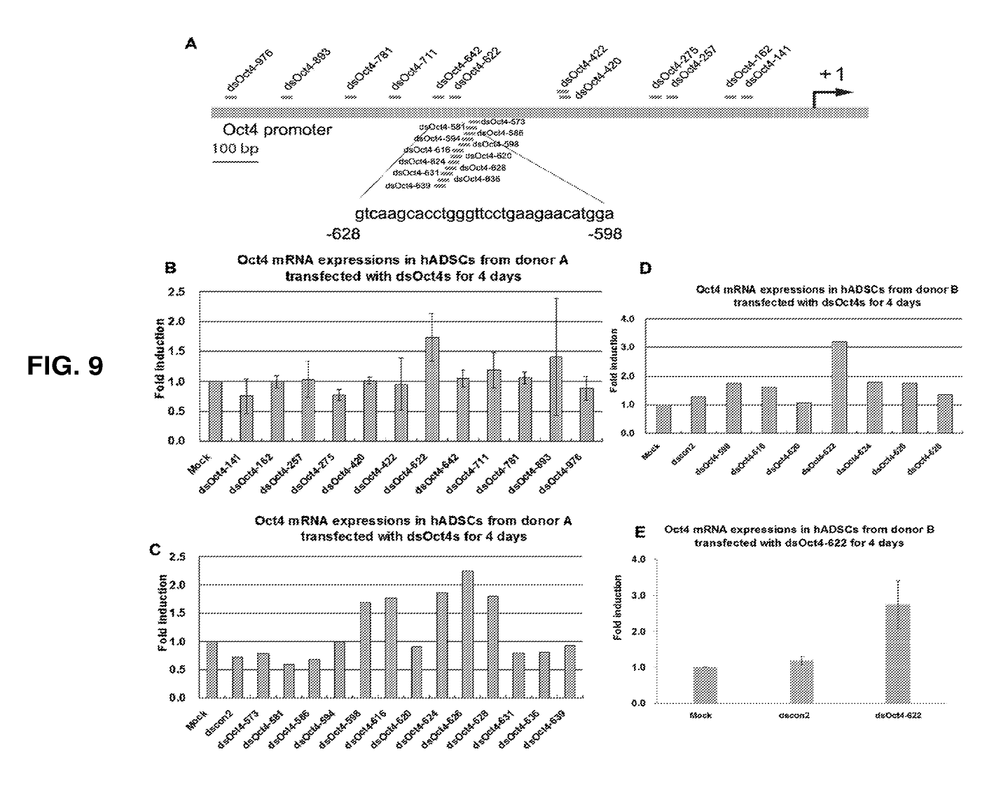

Twelve candidate saRNAs (dsOct4-141, dsOct4-162, dsOct4-257, dsOct4-275, dsOct4-420, dsOct4-422, dsOct4-622, dsOct4-642, dsOct4-711, dsOct4-781, dsOct4-893 and dsOct4-976) that targeted the human Oct4 promoter at sites ranging from −976 to −141 relative to the transcription start site (TSS) were designed (FIG. 9, panel A). Each saRNA was transfected into human adipose-derived stem cells (ADSCs) from donor A and Oct4 expression was evaluated by quantitative real-time RT-PCR (qRT-PCR) 4 days following treatment. Compared to mock, dsOct4-622 induced Oct4 expression by about 1.7-fold (FIG. 9, panel B).

Additional thirteen candidate saRNAs were designed around dsOct4-622 (dsOct4-573, dsOct4-581, dsOct4-586, dsOct4-594, dsOct4-598, dsOct4-616, dsOct4-620, dsOct4-624, dsOct4-626, dsOct4-628, dsOct4-631, dsOct4-636 and dsOct4-639) (FIG. 9, panel A) and individually transfected into human ADSCs from donor A. Four days following transfection, dsOct4-598, dsOct4-616, dsOct4-624, dsOct4-626 and dsOct4-628 induced Oct4 mRNA expression by about 1.7-fold to about 2.2-fold (FIG. 9, panel C).

To determine whether Oct4 is activated b these saRNAs in ADSCs from other donors, dsOct4-598, dsOct4-616, dsOct4-620, dsOct4-622, dsOct4-624, dsOct4-626 and dsOct4-628 were transfected into ADSCs from donor B. Consistent with the results obtained with ADSCs from donor A, most of these saRNAs activated endogenous Oct4 at mRNA levels, with dsOct4-622 inducing Oct4 expression by about 3-fold (FIG. 9, panels D and E).

FIG. 9, panel A. Schematic representation of Oct4 saRNA design. The DNA sequence of a RNAa hotspot between −628 and −598 relative to the TSS is shown at the bottom. FIG. 9, panels B-E. Oct4 mRNA expression analyzed by real-time RT-PCR in ADSCs from donors A and B. Results in FIG. 9, panels B and E are means±SD of at least 3 independent experiments.

Interestingly, the Oct4 promoter appears to have a hot spot at the region ranging from −628 to −598 relative to Oct4 TSS since most targets within this region are responsive to RNAa (FIG. 9, panel A).

### Example 9

### Replacement of Viral Vector Expressing Oct4 by Oct4-Specific saRNA Leads to iPS Cell Derivation

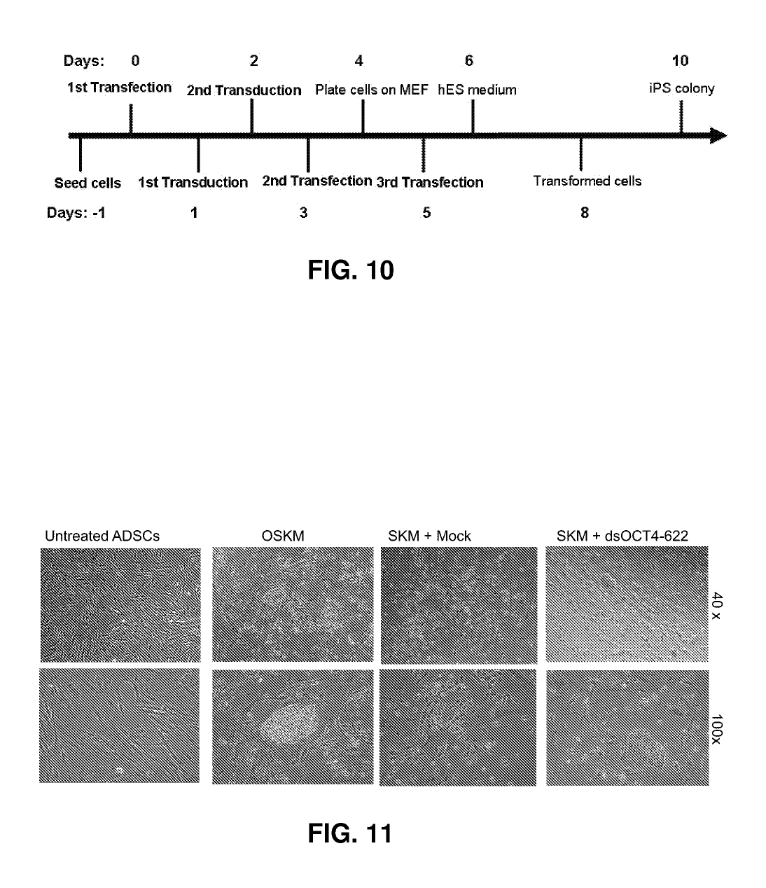

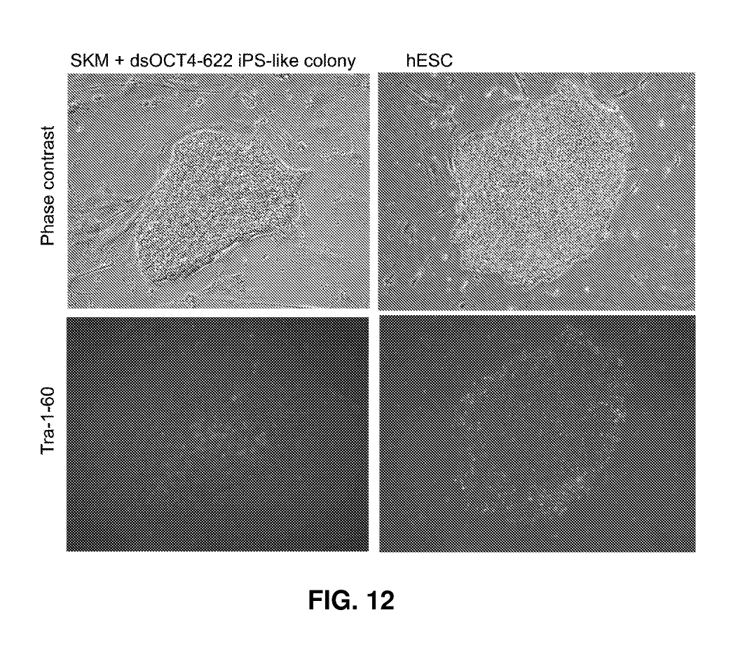

To test whether Oct4 saRNAs identified could functionally replace Oct4 virus in four-factor mediated iPS cell induction, an iPS reprogramming platform in human ADSCs using Yamanaka's retroviral viral iPS system was established. The reprogramming protocol utilized is shown in FIG. 10. Human ADSCs were first mock transfected or transfected with control saRNA (dsControl) or dsOct4-622 on day 0, and then retrovirally transduced on day 1 and 2 with 4 viral factors [Oct4, SOX2, KLF4 and MYC (OSKM)] or 3 factors (SKM). The cells were then transfected with saRNA for two more times on day 3 and day 5. On day 4, the cells were re-plated onto irradiated mouse embryonic fibroblast (MEF) feeder cells and on day 6 cultured in human embryonic stem (hES) cell growth medium. From day 8, transformed cells could be observed in all treatment. On day 9-10, iPS-like colonies started to appear in 4 factors infected cells or cells infected with SKM and transfected with dsOct4-622. No apparent iPS-like colonies appeared in cells that were infected with SKM factors and transfected with control saRNA (FIG. 12). The resulted iPS colonies (OSKM or SKM+dsOct4-622) had typical iPS morphology. They were tightly packed with clearly defined border. Cells in the colonies had a high nuclear-to-cytoplasm ratio with prominent nucleoli. See FIGS. 11 and 12.

FIG. 11. Replacing Oct4 virus resulted in induction of iPS-like colonies. ADSCs were treated as described in FIG. 10. Representative phase contrast images were taken on day 12 following the initial saRNA transfection.

Alkaline Phosphatase (AP) staining is widely used method to identify iPS colonies. Since some types of mesenchymal stem cells express relatively high level of the endogenous AP, AP staining is not suitable for staining ADSC-derived iPS cells. Instead, Tra-1-60 staining on live iPS-like colonies was performed. As shown in FIG. 12, SKM+dsOct4-622 derived iPS-like colonies showed obvious green fluorescence especially in cells located in the middle of the colonies. Cells on the edge of these colonies had weaker fluorescence, probably resulting from incomplete reprogramming and/or spontaneous differentiation following initial iPS induction.

FIG. 12. Tra-1-60 staining of iPS-like colonies. iPS-like colonies derived from SKM+dsOct4-622 treatment on day 16 were stained with anti-Tra-1-60 antibody. Human ES cells served as a positive control.

The observation that replacing Oct4 virus by Oct4 saRNA can generate iPS-like colonies with a similar efficiency as 4 factors (OSKM) was reproducible in at least 3 reprogramming experiments.

That which is claimed is:

1. A method for inducing a somatic cell to form an induced pluripotent stem cell, the method comprising:
introducing into a somatic cell a short activating RNA (saRNA) molecule, wherein the saRNA molecule is up to 30 nucleotides long and comprises a first ribonucleic acid strand comprising a ribonucleic acid sequence at least 80% complementary to a promoter region sequence of a transcription factor gene, wherein the transcription factor gene encodes a transcription factor that induces the somatic cell to form a pluripotent stem cell,
wherein the introducing is sufficient to activate transcription of the transcription factor gene; and
culturing the somatic cell to produce an iPS cell.

2. The method of claim 1, wherein the saRNA molecule comprises a second ribonucleic acid strand comprising a ribonucleic acid sequence at least 80% complementary to the ribonucleic acid of the first ribonucleic acid strand.

3. The method of claim 1, wherein the somatic cell is a human somatic cell.

4. The method of claim 1, wherein the transcription factor gene is Oct4, Sox2, c-Myc, Klf4, Nanog, Lin28 or NR5A2.

5. The method of claim 1, wherein the promoter region is a sequence present between position −628 and −598 relative to Oct4 transcription start site.

6. The method of claim 1, wherein the transcription factor gene is Oct4 and the first ribonucleic acid strand comprises a sequence selected from SEQ ID NO: 1 to SEQ ID NO: 40 and SEQ ID NO: 69 to SEQ ID NO: 108.

7. The method of claim 6, wherein the saRNA molecule comprises a second ribonucleic acid strand at least 80% complementary to the first ribonucleic acid strand.

8. The method of claim 1, wherein the transcription factor gene is c-Myc and the first ribonucleic acid strand comprises a sequence selected from SEQ ID NO: 41-SEQ ID NO: 62 and SEQ ID NO: 109-SEQ ID NO: 130.

9. The method of claim 1, wherein the transcription factor gene is Nanog and the first ribonucleic acid strand comprises a sequence selected from SEQ ID NO: 63-SEQ ID NO: 66 and SEQ ID NO: 131-SEQ ID NO: 134.

10. The method of claim 1, wherein the transcription factor gene is Klf4 and the first ribonucleic acid strand comprises a sequence selected from SEQ ID NO: 67, SEQ ID NO: 68, SEQ ID NO: 135, and SEQ ID NO: 136.

11. The method of claim 1, wherein the introducing comprises introducing into the somatic cell a second saRNA molecule, wherein the second saRNA molecule is up to 30 nucleotides long and comprises a first ribonucleic acid strand comprising a ribonucleic acid sequence at least 80% complementary to a promoter region sequence of a second transcription factor gene, wherein the second transcription factor gene encodes a transcription factor that induces the somatic cell to form a pluripotent stem cell,
wherein the introducing is sufficient to activate transcription of the second transcription factor gene.

12. The method of claim 11, wherein the introducing comprises introducing into the somatic cell a third saRNA molecule, wherein the third saRNA molecule is up to 30 nucleotides long and comprises a first ribonucleic acid strand comprising a ribonucleic acid sequence at least 80% complementary to a promoter region sequence of a third transcription factor gene, wherein the third transcription factor gene encodes a transcription factor that induces the somatic cell to form a pluripotent stem cell,
wherein the introducing is sufficient to activate transcription of the third transcription factor gene.

13. The method of claim 11, wherein the introducing comprises introducing into the somatic cell a fourth saRNA molecule, wherein the fourth saRNA molecule is up to 30 nucleotides long and comprises a first ribonucleic acid strand comprising a ribonucleic acid sequence at least 80% complementary to a promoter region sequence of a fourth transcription factor gene, wherein the fourth transcription factor gene encodes a transcription factor that induces the somatic cell to form a pluripotent stem cell,
wherein the introducing is sufficient to activate transcription of the fourth transcription factor gene.

14. The method of claim 1, wherein the introducing comprises introducing into the somatic cell a nucleic acid encoding a second transcription factor that induces the somatic cell to form a pluripotent stem cell.

15. The method of claim 1, wherein the introducing comprises introducing into the somatic cell a plurality of nucleic acids encoding a plurality of transcription factors that induce the somatic cell to form a pluripotent stem cell.

16. The method of claim 1, wherein the ribonucleic acid sequence is at least 85% complementary to the promoter region sequence.

17. The method of claim 1, wherein the ribonucleic acid sequence is at least 90% complementary to the promoter region sequence.

18. The method of claim 1, wherein the ribonucleic acid sequence is at least 95% complementary to the promoter region sequence.

19. The method of claim 1, wherein the ribonucleic acid sequence is at least 98% complementary to the promoter region sequence.

20. An isolated composition comprising,
a saRNA molecule up to 30 nucleotides long and comprising a first ribonucleic acid strand comprising a ribonucleic acid sequence at least 80% identical to a sequence selected from SEQ ID NO: 1 to SEQ ID NO: 40 and SEQ ID NO: 69 to SEQ ID NO: 108, wherein the sequence is sufficient to activate transcription of the Oct4 gene.

21. The composition of claim 20, wherein the saRNA molecule comprises a second ribonucleic acid strand at least 80% complementary to the first ribonucleic acid strand.

22. The composition of claim 20, wherein the ribonucleic acid sequence is at least 85% identical to a sequence selected from SEQ ID NO: 1 to SEQ ID NO: 40 and SEQ ID NO: 69 to SEQ ID NO: 108.

23. The composition of claim 20, wherein the ribonucleic acid sequence is at least 90% identical to a sequence selected from SEQ ID NO: 1 to SEQ ID NO: 40 and SEQ ID NO: 69 to SEQ ID NO: 108.

24. The composition of claim 20, wherein the ribonucleic acid sequence is at least 95% identical to a sequence selected from SEQ ID NO: 1 to SEQ ID NO: 40 and SEQ ID NO: 69 to SEQ ID NO: 108.

25. An isolated composition comprising,
a saRNA molecule up to 30 nucleotides long and comprising at least a first ribonucleic acid strand comprising a sequence at least 80% identical to a sequence selected from SEQ ID NO: 63-SEQ ID NO: 66 and SEQ ID NO: 131-SEQ ID NO: 134, wherein the sequence is sufficient to activate transcription of the Nanog gene.

26. The composition of claim 25, wherein the saRNA molecule comprises a second ribonucleic acid strand at least 80% complementary to the first ribonucleic acid strand.

27. The composition of claim 25, wherein the composition comprises a second saRNA molecule comprising at least one ribonucleic acid strand, wherein when the first ribonucleic acid strand comprises a sequence at least 80% identical to the sequence of SEQ ID NO: 63, the one ribonucleic acid strand of the second saRNA comprises a sequence at least 80% identical to the sequence of SEQ ID NO: 65 or SEQ ID NO: 66.

28. The composition of claim 25, wherein the ribonucleic acid sequence is at least 85% identical to a sequence selected from SEQ ID NO: 63-SEQ ID NO: 66 and SEQ ID NO: 131-SEQ ID NO: 134.

29. The composition of claim 25, wherein the ribonucleic acid sequence is at least 90% identical to a sequence selected from SEQ ID NO: 63-SEQ ID NO: 66 and SEQ ID NO: 131-SEQ ID NO: 134.

30. The composition of claim 25, wherein the ribonucleic acid sequence is at least 95% identical to a sequence selected from SEQ ID NO: 63-SEQ ID NO: 66 and SEQ ID NO: 131-SEQ ID NO: 134.

31. An isolated composition comprising,
a saRNA molecule up to 30 nucleotides long and comprising a first ribonucleic acid strand comprising a ribonucleic acid sequence at least 80% identical to a sequence selected from SEQ ID NO: 41-SEQ ID NO: 62 and SEQ ID NO: 109-SEQ ID NO: 130, wherein the sequence is sufficient to activate transcription of the c-Myc gene.

32. The composition of claim 31, wherein the ribonucleic acid sequence is at least 85% identical to a sequence selected from SEQ ID NO: 41-SEQ ID NO: 62 and SEQ ID NO: 109-SEQ ID NO: 130.

33. The composition of claim 31, wherein the ribonucleic acid sequence is at least 90% identical to a sequence selected from SEQ ID NO: 41-SEQ ID NO: 62 and SEQ ID NO: 109-SEQ ID NO: 130.

34. The composition of claim 31, wherein the ribonucleic acid sequence is at least 95% identical to a sequence selected from SEQ ID NO: 41-SEQ ID NO: 62 and SEQ ID NO: 109-SEQ ID NO: 130.

35. The composition of claim 31, wherein the ribonucleic acid sequence is at least 98% identical to a sequence selected from SEQ ID NO: 41-SEQ ID NO: 62 and SEQ ID NO: 109-SEQ ID NO: 130.

36. An iPS cell comprising at least one exogenous saRNA molecule up to 30 nucleotides long, wherein the saRNA molecule comprises a first ribonucleic acid strand comprising a ribonucleic acid sequence at least 80% complementary to a promoter region sequence of a transcription factor gene, wherein the transcription factor gene encodes a transcription factor that induces a somatic cell to form a pluripotent stem cell.

37. The method of claim 36, wherein the saRNA molecule comprises a second ribonucleic acid strand comprising a ribonucleic acid sequence complementary to the ribonucleic acid of the first acid strand.

38. The method of claim 36, wherein the transcription factor gene is Oct4, Sox2, c-Myc, Klf4, Nanog, Lin28 or NR5A2.


SEQUENCE LISTING (139 sequences)

SEQ ID NO: 1 - RNA, 19 nt, Artificial Sequence
  synthetic nucleic acid sequence
gccaccaccauuaggcaaa

SEQ ID NO: 2 - RNA, 19 nt, Artificial Sequence
  synthetic nucleic acid sequence
uuugccuaauggugguggc

SEQ ID NO: 3 - RNA, 19 nt, Artificial Sequence
  synthetic nucleic acid sequence
uccccuuccacagacacca

SEQ ID NO: 4 - RNA, 19 nt, Artificial Sequence
  synthetic nucleic acid sequence
uggugucuguggaagggga

SEQ ID NO: 5 - RNA, 19 nt, Artificial Sequence
  synthetic nucleic acid sequence
agugagacccugucuuaaa

SEQ ID NO: 6 - RNA, 19 nt, Artificial Sequence
  synthetic nucleic acid sequence
uuuaagacagggucucacu

SEQ ID NO: 7 - RNA, 19 nt, Artificial Sequence
  synthetic nucleic acid sequence
cuccagucugggcaacaaa

SEQ ID NO: 8 - RNA, 19 nt, Artificial Sequence
  synthetic nucleic acid sequence
uuuguugcccagacuggag

SEQ ID NO: 9 - RNA, 19 nt, Artificial Sequence
  synthetic nucleic acid sequence
ggccccaucucuacuaaaa

SEQ ID NO: 10 - RNA, 19 nt, Artificial Sequence
  synthetic nucleic acid sequence
uuuuaguagagauggggcc

SEQ ID NO: 11 - RNA, 19 nt, Artificial Sequence
  synthetic nucleic acid sequence
aaggccccaucucuacuaa

SEQ ID NO: 12 - RNA, 19 nt, Artificial Sequence
  synthetic nucleic acid sequence
uuaguagagauggggccuu

SEQ ID NO: 13 - RNA, 19 nt, Artificial Sequence
  synthetic nucleic acid sequence
ugggaugugcagagccuga

SEQ ID NO: 14 - RNA, 19 nt, Artificial Sequence
  synthetic nucleic acid sequence
ucaggcucugcacauccca

SEQ ID NO: 15 - RNA, 19 nt, Artificial Sequence
  synthetic nucleic acid sequence
cagacagcugggaugugca

SEQ ID NO: 16 - RNA, 19 nt, Artificial Sequence
  synthetic nucleic acid sequence
ugcacaucccagcugucug

SEQ ID NO: 17 - RNA, 19 nt, Artificial Sequence
  synthetic nucleic acid sequence
gauuccagacagcugggau

SEQ ID NO: 18 - RNA, 19 nt, Artificial Sequence
  synthetic nucleic acid sequence
aucccagcugucuggaauc

SEQ ID NO: 19 - RNA, 19 nt, Artificial Sequence
  synthetic nucleic acid sequence
gugggagugauuccagaca

SEQ ID NO: 20 - RNA, 19 nt, Artificial Sequence
  synthetic nucleic acid sequence
ugucuggaaucacucccac

SEQ ID NO: 21 - RNA, 19 nt, Artificial Sequence
  synthetic nucleic acid sequence
aggugugggagugauucca

SEQ ID NO: 22 - RNA, 19 nt, Artificial Sequence
  synthetic nucleic acid sequence
uggaaucacucccacaccu

SEQ ID NO: 23 - RNA, 19 nt, Artificial Sequence
  synthetic nucleic acid sequence
gguuccugaagaacaugga

SEQ ID NO: 24 - RNA, 19 nt, Artificial Sequence
  synthetic nucleic acid sequence
uccauguucuucaggaacc

SEQ ID NO: 25 - RNA, 19 nt, Artificial Sequence
  synthetic nucleic acid sequence
ccuggguuccugaagaaca

SEQ ID NO: 26 - RNA, 19 nt, Artificial Sequence
  synthetic nucleic acid sequence
uguucuucaggaacccagg

SEQ ID NO: 27 - RNA, 19 nt, Artificial Sequence
  synthetic nucleic acid sequence
caccuggguuccugaagaa

SEQ ID NO: 28 - RNA, 19 nt, Artificial Sequence
  synthetic nucleic acid sequence
uucuucaggaacccaggug

SEQ ID NO: 29 - RNA, 19 nt, Artificial Sequence
  synthetic nucleic acid sequence
aagcaccuggguuccugaa

SEQ ID NO: 30 - RNA, 19 nt, Artificial Sequence
  synthetic nucleic acid sequence
uucaggaacccaggugcuu

SEQ ID NO: 31 - RNA, 19 nt, Artificial Sequence
  synthetic nucleic acid sequence
caagcaccuggguuccuga

SEQ ID NO: 32 - RNA, 19 nt, Artificial Sequence
  synthetic nucleic acid sequence
ucaggaacccaggugcuug

SEQ ID NO: 33 - RNA, 19 nt, Artificial Sequence
  synthetic nucleic acid sequence
gucaagcaccuggguuccu

SEQ ID NO: 34 - RNA, 19 nt, Artificial Sequence
  synthetic nucleic acid sequence
aggaacccaggugcuugac

SEQ ID NO: 35 - RNA, 19 nt, Artificial Sequence
  synthetic nucleic acid sequence
ggggucaagcaccuggguu

SEQ ID NO: 36 - RNA, 19 nt, Artificial Sequence
  synthetic nucleic acid sequence
aacccaggugcuugacccc

SEQ ID NO: 37 - RNA, 19 nt, Artificial Sequence
  synthetic nucleic acid sequence
gagagggggucaagcaccu

SEQ ID NO: 38 - RNA, 19 nt, Artificial Sequence
  synthetic nucleic acid sequence
aggugcuugacccccucuc

SEQ ID NO: 39 - RNA, 19 nt, Artificial Sequence
  synthetic nucleic acid sequence
guggagagggggucaagca

SEQ ID NO: 40 - RNA, 19 nt, Artificial Sequence
  synthetic nucleic acid sequence
ugcuugacccccucuccac

SEQ ID NO: 41 - RNA, 19 nt, Artificial Sequence
  synthetic nucleic acid sequence
gcauacauaaugcauaaua

SEQ ID NO: 42 - RNA, 19 nt, Artificial Sequence
  synthetic nucleic acid sequence
uauuaugcauuauguaugc

SEQ ID NO: 43 - RNA, 19 nt, Artificial Sequence
  synthetic nucleic acid sequence
gacacaucucagggcuaaa

SEQ ID NO: 44 - RNA, 19 nt, Artificial Sequence
  synthetic nucleic acid sequence
uuuagcccugagauguguc

SEQ ID NO: 45 - RNA, 19 nt, Artificial Sequence
  synthetic nucleic acid sequence
ucugcugcuuuggcagcaa

SEQ ID NO: 46 - RNA, 19 nt, Artificial Sequence
  synthetic nucleic acid sequence
uugcugccaaagcagcaga

SEQ ID NO: 47 - RNA, 19 nt, Artificial Sequence
  synthetic nucleic acid sequence
gauagcugugcauacauaa

SEQ ID NO: 48 - RNA, 19 nt, Artificial Sequence
  synthetic nucleic acid sequence
uuauguaugcacagcuauc

SEQ ID NO: 49 - RNA, 19 nt, Artificial Sequence
  synthetic nucleic acid sequence
gcauacauaaugcauaaua

SEQ ID NO: 50 - RNA, 19 nt, Artificial Sequence
  synthetic nucleic acid sequence
uauuaugcauuauguaugc

SEQ ID NO: 51 - RNA, 19 nt, Artificial Sequence
  synthetic nucleic acid sequence
gcccauuaauacccuucuu

SEQ ID NO: 52 - RNA, 19 nt, Artificial Sequence
  synthetic nucleic acid sequence
aagaaggguauuaaugggc

SEQ ID NO: 53 - RNA, 19 nt, Artificial Sequence
  synthetic nucleic acid sequence
ggcccuuuccccagccuua

SEQ ID NO: 54 - RNA, 19 nt, Artificial Sequence
  synthetic nucleic acid sequence
uaaggcuggggaaagggcc

SEQ ID NO: 55 - RNA, 19 nt, Artificial Sequence
  synthetic nucleic acid sequence
caaggaugcgguuugucaa

SEQ ID NO: 56 - RNA, 19 nt, Artificial Sequence
  synthetic nucleic acid sequence
uugacaaaccgcauccuug

SEQ ID NO: 57 - RNA, 19 nt, Artificial Sequence
  synthetic nucleic acid sequence
ugcgguuugucaaacagua

SEQ ID NO: 58 - RNA, 19 nt, Artificial Sequence
  synthetic nucleic acid sequence
uacuguuugacaaaccgca

SEQ ID NO: 59 - RNA, 19 nt, Artificial Sequence
  synthetic nucleic acid sequence
aggguuugagagggagcaa

SEQ ID NO: 60 - RNA, 19 nt, Artificial Sequence
  synthetic nucleic acid sequence
uugcucccucucaaacccu

SEQ ID NO: 61 - RNA, 19 nt, Artificial Sequence
  synthetic nucleic acid sequence
acucuguuuacauccuaga

SEQ ID NO: 62 - RNA, 19 nt, Artificial Sequence
  synthetic nucleic acid sequence
ucuaggauguaaacagagu

SEQ ID NO: 63 - RNA, 19 nt, Artificial Sequence
  synthetic nucleic acid sequence
gccagauuuugagacacua

SEQ ID NO: 64 - RNA, 19 nt, Artificial Sequence
  synthetic nucleic acid sequence
uagugucucaaaaucuggc

SEQ ID NO: 65 - RNA, 19 nt, Artificial Sequence
  synthetic nucleic acid sequence
gggauagacaagaaaccaa

SEQ ID NO: 66 - RNA, 19 nt, Artificial Sequence
  synthetic nucleic acid sequence
uugguuucuugucuauccc

SEQ ID NO: 67 - DNA, 19 bp, Artificial Sequence
  synthetic nucleic acid sequence
gaacccagggagccgacaa

SEQ ID NO: 68 - RNA, 19 nt, Artificial Sequence
  synthetic nucleic acid sequence
uugucggcucccuggguuc

SEQ ID NO: 69 - DNA, 21 bp, Artificial Sequence
  synthetic nucleic acid sequence. The sequence is a combined DNA/RNA sequence
gccaccaccauuaggcaaatt

SEQ ID NO: 70 - DNA, 21 bp, Artificial Sequence
  synthetic nucleic acid sequence. The sequence is a combined DNA/RNA sequence
uuugccuaauggugguggctt

SEQ ID NO: 71 - DNA, 21 bp, Artificial Sequence
  synthetic nucleic acid sequence. The sequence is a combined DNA/RNA sequence
uccccuuccacagacaccatt

SEQ ID NO: 72 - DNA, 21 bp, Artificial Sequence
  synthetic nucleic acid sequence. The sequence is a combined DNA/RNA sequence
uggugucuguggaaggggatt

SEQ ID NO: 73 - DNA, 21 bp, Artificial Sequence
  synthetic nucleic acid sequence. The sequence is a combined DNA/RNA sequence
agugagacccugucuuaaatt

SEQ ID NO: 74 - DNA, 21 bp, Artificial Sequence
  synthetic nucleic acid sequence. The sequence is a combined DNA/RNA sequence
uuuaagacagggucucacutt

SEQ ID NO: 75 - DNA, 21 bp, Artificial Sequence
  synthetic nucleic acid sequence. The sequence is a combined DNA/RNA sequence
cuccagucugggcaacaaatt

SEQ ID NO: 76 - DNA, 21 bp, Artificial Sequence
  synthetic nucleic acid sequence. The sequence is a combined DNA/RNA sequence
uuuguugcccagacuggagtt

SEQ ID NO: 77 - DNA, 21 bp, Artificial Sequence
  synthetic nucleic acid sequence. The sequence is a combined DNA/RNA sequence
ggccccaucucuacuaaaatt

SEQ ID NO: 78 - DNA, 21 bp, Artificial Sequence
  synthetic nucleic acid sequence. The sequence is a combinded DNA/RNA sequence
uuuuaguagagauggggcctt

SEQ ID NO: 79 - DNA, 21 bp, Artificial Sequence
  synthetic nucleic acid sequence. The sequence is a combined DNA/RNA sequence
aaggccccaucucuacuaatt

SEQ ID NO: 80 - DNA, 21 bp, Artificial Sequence
  synthetic nucleic acid sequence. The sequence is a combined DNA/RNA sequence
uuaguagagauggggccuutt

SEQ ID NO: 81 - DNA, 21 bp, Artificial Sequence
  synthetic nucleic acid sequence. The sequence is a combined DNA/RNA sequence
ugggaugugcagagccugatt

SEQ ID NO: 82 - DNA, 21 bp, Artificial Sequence
  synthetic nucleic acid sequence. The sequence is a combined DNA/RNA sequence
ucaggcucugcacaucccatt

SEQ ID NO: 83 - DNA, 21 bp, Artificial Sequence
  synthetic nucleic acid sequence. The sequence is a combinded DNA/RNA sequence
cagacagcugggaugugcatt

SEQ ID NO: 84 - DNA, 21 bp, Artificial Sequence
  synthetic nucleic acid sequence. The sequence is a combined DNA/RNA sequence
ugcacaucccagcugucugtt

SEQ ID NO: 85 - DNA, 21 bp, Artificial Sequence
  synthetic nucleic acid sequence. The sequence is a combined DNA/RNA sequence
gauuccagacagcugggautt

SEQ ID NO: 86 - DNA, 21 bp, Artificial Sequence
  synthetic nucleic acid sequence. The sequence is a combined DNA/RNA sequence
aucccagcugucuggaauctt

SEQ ID NO: 87 - DNA, 21 bp, Artificial Sequence
  synthetic nucleic acid sequence. The sequence is a combined DNA/RNA sequence
gugggagugauuccagacatt

SEQ ID NO: 88 - DNA, 21 bp, Artificial Sequence
  synthetic nucleic acid sequence. The sequence is a combined DNA/RNA sequence
ugucuggaaucacucccactt

SEQ ID NO: 89 - DNA, 21 bp, Artificial Sequence
  synthetic nucleic acid sequence. The sequence is a combined DNA/RNA sequence
aggugugggagugauuccatt

SEQ ID NO: 90 - DNA, 21 bp, Artificial Sequence
  synthetic nucleic acid sequence. The sequence is a combined DNA/RNA sequence
uggaaucacucccacaccutt

SEQ ID NO: 91 - DNA, 21 bp, Artificial Sequence
  The sequence is a DNA/RNA sequence
gguuccugaagaacauggatt

SEQ ID NO: 92 - DNA, 21 bp, Artificial Sequence
  synthetic nucleic acid sequence. The sequence is a combinded DNA/RNA sequence
uccauguucuucaggaacctt

SEQ ID NO: 93 - DNA, 21 bp, Artificial Sequence
  synthetic nucleic acid sequence. The sequence is a combined DNA/RNA sequence
ccuggguuccugaagaacatt

SEQ ID NO: 94 - DNA, 21 bp, Artificial Sequence
  synthetic nucleic acid sequence. The sequence is a combined DNA/RNA sequence
uguucuucaggaacccaggtt

SEQ ID NO: 95 - DNA, 21 bp, Artificial Sequence
  synthetic nucleic acid sequence. The sequence is a combined DNA/RNA sequence
caccuggguuccugaagaatt

SEQ ID NO: 96 - DNA, 21 bp, Artificial Sequence
  synthetic nucleic acid sequence. The sequence is a combined DNA/RNA sequence
uucuucaggaacccaggugtt

SEQ ID NO: 97 - DNA, 21 bp, Artificial Sequence
  synthetic nucleic acid sequence. The sequence is a combined DNA/RNA sequence
aagcaccuggguuccugaatt

SEQ ID NO: 98 - DNA, 21 bp, Artificial Sequence
  synthetic nucleic acid sequence. The sequence is a combined DNA/RNA sequence
uucaggaacccaggugcuutt

SEQ ID NO: 99 - DNA, 21 bp, Artificial Sequence
  synthetic nucleic acid sequence. The sequence is a combined DNA/RNA sequence
caagcaccuggguuccugatt

SEQ ID NO: 100 - DNA, 21 bp, Artificial Sequence
  synthetic nucleic acid sequence. The sequence is a combined DNA/RNA sequence
ucaggaacccaggugcuugtt

SEQ ID NO: 101 - DNA, 21 bp, Artificial Sequence
  synthetic nucleic acid sequence. The sequence is a combined DNA/RNA sequence
gucaagcaccuggguuccutt

SEQ ID NO: 102 - DNA, 21 bp, Artificial Sequence
  synthetic nucleic acid sequence. The sequence is a combined DNA/RNA sequence
aggaacccaggugcuugactt

SEQ ID NO: 103 - DNA, 21 bp, Artificial Sequence
  synthetic nucleic acid sequence. The sequence is a combined DNA/RNA sequence
ggggucaagcaccuggguutt

SEQ ID NO: 104 - DNA, 21 bp, Artificial Sequence
  synthetic nucleic acid sequence. The sequence is a combined DNA/RNA sequence
aacccaggugcuugacccctt

SEQ ID NO: 105 - DNA, 21 bp, Artificial Sequence
  synthetic nucleic acid sequence. The sequence is a combined DNA/RNA sequence
gagagggggucaagcaccutt

SEQ ID NO: 106 - DNA, 21 bp, Artificial Sequence
  synthetic nucleic acid sequence. The sequence is a combined DNA/RNA sequence
aggugcuugacccccucuctt

SEQ ID NO: 107 - DNA, 21 bp, Artificial Sequence
  synthetic nucleic acid sequence. The sequence is a combined DNA/RNA sequence
guggagagggggucaagcatt

SEQ ID NO: 108 - DNA, 21 bp, Artificial Sequence
  synthetic nucleic acid sequence. The sequence is a combinded DNA/RNA sequence
ugcuugacccccucuccactt

SEQ ID NO: 109 - DNA, 21 bp, Artificial Sequence
  synthetic nucleic acid sequence. The sequence is a combined DNA/RNA sequence
gcauacauaaugcauaauatt

SEQ ID NO: 110 - DNA, 21 bp, Artificial Sequence
  synthetic nucleic acid sequence. The sequence is a combined DNA/RNA sequence
uauuaugcauuauguaugctt

SEQ ID NO: 111 - DNA, 21 bp, Artificial Sequence
  synthetic nucleic acid sequence. The sequence is a combinded DNA/RNA sequence
gacacaucucagggcuaaatt

SEQ ID NO: 112 - DNA, 21 bp, Artificial Sequence
  synthetic nucleic acid sequence. The sequence is a combinded DNA/RNA sequence
uuuagcccugagauguguctt

SEQ ID NO: 113 - DNA, 21 bp, Artificial Sequence
  synthetic nucleic acid sequence. The sequence is a combinded DNA/RNA sequence
ucugcugcuuuggcagcaatt

SEQ ID NO: 114 - DNA, 21 bp, Artificial Sequence
  synthetic nucleic acid sequence. The sequence is a combinded DNA/RNA sequence
uugcugccaaagcagcagatt

SEQ ID NO: 115 - DNA, 21 bp, Artificial Sequence
  synthetic nucleic acid sequence. The sequence is a combinded DNA/RNA sequence
gauagcugugcauacauaatt

SEQ ID NO: 116 - DNA, 21 bp, Artificial Sequence
  synthetic nucleic acid sequence. The sequence is a combinded DNA/RNA sequence
gauagcugugcauacauaatt

SEQ ID NO: 117 - DNA, 21 bp, Artificial Sequence
  synthetic nucleic acid sequence. The sequence is a combinded DNA/RNA sequence
gcauacauaaugcauaauatt

SEQ ID NO: 118 - DNA, 21 bp, Artificial Sequence
  synthetic nucleic acid sequence. The sequence is a combinded DNA/RNA sequence
uauuaugcauuauguaugctt

SEQ ID NO: 119 - DNA, 21 bp, Artificial Sequence
  synthetic nucleic acid sequence. The sequence is a combined DNA/RNA sequence
gcccauuaauacccuucuutt

SEQ ID NO: 120 - DNA, 21 bp, Artificial Sequence
  synthetic nucleic acid sequence. The sequence is a combinded DNA/RNA sequence
aagaaggguauuaaugggctt

SEQ ID NO: 121 - DNA, 21 bp, Artificial Sequence
  synthetic nucleic acid sequence. The sequence is a combined DNA/RNA sequence
ggcccuuuccccagccuuatt

SEQ ID NO: 122 - DNA, 21 bp, Artificial Sequence
  synthetic nucleic acid sequence. The sequence is a combinded DNA/RNA sequence
uaaggcuggggaaagggcctt

SEQ ID NO: 123 - DNA, 21 bp, Artificial Sequence
  synthetic nucleic acid sequence. The sequence is a combinded DNA/RNA sequence
caaggaugcgguuugucaatt

SEQ ID NO: 124 - DNA, 21 bp, Artificial Sequence
  synthetic nucleic acid sequence. The sequence is a combinded DNA/RNA sequence
uugacaaaccgcauccuugtt

SEQ ID NO: 125 - DNA, 21 bp, Artificial Sequence
  synthetic nucleic acid sequence. The sequence is a combined DNA/RNA sequence
ugcgguuugucaaacaguatt

SEQ ID NO: 126 - DNA, 21 bp, Artificial Sequence
  synthetic nucleic acid sequence. The sequence is a combined DNA/RNA sequence
uacuguuugacaaaccgcatt

SEQ ID NO: 127 - DNA, 21 bp, Artificial Sequence
  synthetic nucleic acid sequence. The sequence is a combinded DNA/RNA sequence
aggguuugagagggagcaatt

SEQ ID NO: 128 - DNA, 21 bp, Artificial Sequence
  synthetic nucleic acid sequence. The sequence is a combined DNA/RNA sequence
uugcucccucucaaacccutt

SEQ ID NO: 129 - DNA, 21 bp, Artificial Sequence
  synthetic nucleic acid sequence. The sequence is a combinded DNA/RNA sequence
acucuguuuacauccuagatt

SEQ ID NO: 130 - DNA, 21 bp, Artificial Sequence
  synthetic nucleic acid sequence. The sequence is a combined DNA/RNA sequence
ucuaggauguaaacagagutt

SEQ ID NO: 131 - DNA, 21 bp, Artificial Sequence
  synthetic nucleic acid sequence. The sequence is a combined DNA/RNA sequence
gccagauuuugagacacuatt

SEQ ID NO: 132 - DNA, 21 bp, Artificial Sequence
  synthetic nucleic acid sequence. The sequence is a combined DNA/RNA sequence
uagugucucaaaaucuggctt

SEQ ID NO: 133 - DNA, 21 bp, Artificial Sequence
  synthetic nucleic acid sequence. The sequence is a combined DNA/RNA sequence
gggauagacaagaaaccaatt

SEQ ID NO: 134 - DNA, 21 bp, Artificial Sequence
  synthetic nucleic acid sequence. The sequence is a combined DNA/RNA sequence
uugguuucuugucuauccctt

SEQ ID NO: 135 - DNA, 21 bp, Artificial Sequence
  synthetic nucleic acid sequence. The sequence is a combined DNA/RNA sequence
gaacccagggagccgacaatt

SEQ ID NO: 136 - DNA, 21 bp, Artificial Sequence
  synthetic nucleic acid sequence. The sequence is a combined DNA/RNA sequence
uugucggcucccuggguuctt

SEQ ID NO: 137 - DNA, 31 bp, Homo Sapiens
gtcaagcacctgggttcctgaagaacatgga

SEQ ID NO: 138 - DNA, 19 bp, Artificial Sequence
  synthetic oligonucleotide
tcccttcgcaagccctcat

SEQ ID NO: 139 - DNA, 31 bp, Artificial Sequence
  synthetic oligonucleotide
tgacggtgcagggctccggggaggccccatc